(19) United States
(12) Patent Application Publication
(10) Pub. No.: US 2021/0207347 A1
(43) Pub. Date: Jul. 8, 2021

(54) Method and Apparatus for Delivering Temperature Controlled Water

(71) Applicants: FAWAZ SALIM SALEEM, LOMA LINDA, CA (US); CHARLES OBICHANG, REDLANDS, CA (US)

(72) Inventors: FAWAZ SALIM SALEEM, LOMA LINDA, CA (US); CHARLES OBICHANG, REDLANDS, CA (US)

(21) Appl. No.: 17/126,550

(22) Filed: Dec. 18, 2020

Related U.S. Application Data
(62) Division of application No. 16/786,909, filed on Feb. 10, 2020, now abandoned.

(51) Int. Cl. E03C 1/04; A61L 2/26; A61L 2/00; E03C 1/046

(52) U.S. Cl. CPC E03C 1/041; A61L 2/26; A61L 2/0088; E03C 2001/0418; A61L 2202/15; A61L 2202/17; E03C 1/046

(57) ABSTRACT

A method and apparatus for delivering water to a user at a control temperature by receiving cold water, receiving hot water and then adjusting a hot-side graduated-valve and a cold-side graduated-valve in order to adjust the temperature of mixed water according to an initial target temperature value. The temperature of the mixed water is sensed in order to provide feedback, which is necessary to adjust the hot-side graduated valve and the cold-side graduated valve. In some embodiments, hot-side graduated valve and the cold-site graduate-valve are also adjusted to achieve a target flow rate. A target temperature and a target flow rate is communicated to the apparatus by way of an air-gesture sensor.

### RELATED APPLICATIONS

The present application is a divisional of and claims priority to U.S. patent application Ser. No. 16/786,909, entitled “METHOD AND APPARATUS FOR DELIVERING TEMPERATURE CONTROLLED WATER” by Saleem et al., which was filed on Feb. 10, 2020 the text and drawings of which are incorporated by reference into this application in their entirety.

### BACKGROUND

Modern surgical techniques are highly successful due in no small part to effective control of infection. Controlling infection, in turn, requires sterilization of surgical instruments before an operation takes place. Also, it is important that stray infectious matter is neutralized, or the potential of infection from such stray infectious matter is substantially eliminated. Collectively, all of the techniques used in mitigating the potential for infection are generally referred to as “infection control”.

Most people outside of the medical community realize that is important to sterilize instruments. Also, laypersons appreciate that everyone in the surgical theater is attired in substantially sterile garments and wear gloves that form a protective barrier between the surgeon and the patient. Laypersons also understand that, as a result of fictional depictions in movies and television, the surgeon and other staff entering the surgical theater “scrub up” before putting on their sterilized gloves.

A surgical scrub is performed in order to remove resident and transient microorganisms from the hands. It is also important to inhibit the re-growth of flora for the duration of the surgical procedure. By inhibiting such re-growth, there is an added safety for the patient in the event that the glove is somehow compromised during surgery. In other words, should the glove be torn, or accidentally cut, there is less likelihood of transfer of microbial infection to the patient when flora normally resident on the hands is substantially prevented from multiplying. And, according to the World Health Organization's guidelines for hand hygiene, 35% of all gloves have been punctured after just two hours of surgery. Certainly, there is great motivation in inhibiting regrowth of flora.

Amazingly, what is an effective washing of the hands prior to performing a surgical procedure is still widely debated. For example, there are proponents of antimicrobial surgical scrub solutions. In an ordinary environment, these are typically known as hand sanitizers. Amongst the community of surgical professionals, such surgical scrub solutions are known as handrub formulations. As one might expect, proponents of antimicrobial surgical scrub solutions also include the manufacturers of such products. In general, it is the persisting effect that antimicrobial surgical scrub solutions purportedly offered by such products is a compelling argument for preventing resurgence of flora on a surgeon's hands, especially when the hands are in a warm environment formed between the glove and the skin itself.

As compelling as the arguments may be, most people, including professional hospital practitioners and surgeons, still see the need for a prolonged agitation of the skin under running water. In other words, surgeons do and will continue to prefer aggressive washing of the hands using antibacterial soap and hot running water. Commonly used antisepsis agents include chlorhexidine and povidone-iodine.

In its guidelines for hand hygiene, the World Health Organization (“WHO”) indicates that has previously recommended various hand formulations. It also admits that these handrub formulations, as tested by two independent laboratories, failed to pass antisepsis requirements. Accordingly, the WHO acknowledges that further research is necessary because there is still not enough information or antidotal data regarding the use of the handrub formulations that it itself has recommended. So, the use of water and antibacterial soaps and antisepsis agents will continue to be a mainstay of surgical hand preparation.

The proper technique for the use of water and soap in preparing for surgery has also evolved over the years. For example, in 1834, preparation for surgery included three steps. First, the hands were to be washed with hot water and medicated soap for at least five minutes. Then, a 90% ethanol solution was to be applied for a period of 3 to 5 minutes and finally, the hands are to be rinsed with an antiseptic liquid. In 1939, a seven-minute hand wash with soap and water was to be followed by a 70% ethanol mixture for three minutes, but after drying the hands with a towel. Today, most healthcare institutions require a five-minute handwashing regimen. Even still, there is wide variation in the amount of time dedicated to a particular washing practice and even the temperature of the water that must be used.

Surgeons are just as prone to error as any ordinary human being. However, when ordinary people fail to wash their hands properly, patients are not placed at risk. However, should a surgeon be distracted during “scrubbing”, a patient stands the risk of severe infection as a result of what would ordinarily be a simple surgical procedure. And, it is also interesting to appreciate that the exact technique for handwashing can vary based on the type and duration of surgical procedure intended.

### BRIEF DESCRIPTION OF THE DRAWINGS

Several alternative embodiments will hereinafter be described in conjunction with the appended drawings and figures, wherein like numerals denote like elements, and in which:

FIGS. 1A and 1B constitute a flow diagram that depicts one example method for delivering temperature controlled water;

FIG. 2 is a flow diagram that depicts one variation of the present method wherein a user is allowed to provide a temperature-directive;

FIG. 3 is a flow diagram one alternative example method that includes steps for a temperature safety cut-off for an incoming hot water stream;

FIG. 4 is a flow diagram that depicts one variation of the present method wherein characteristics of an incoming stream of cold water are monitored in order to mitigate the potential for scalding water from reaching a user;

FIG. 5 is a flow diagram that illustrates yet another variation of the present method for avoiding application of scalding water based on the temperature of the mixed water;

FIG. 6 is a flow diagram that depicts an alternative variation of the present method wherein characteristics of a surgical scrub cycle are recorded for compliance confirmation purposes;

FIG. 7 is a flow diagram that depicts a variation of the present method wherein the amount of time spent by a user washing their hands is recorded;

FIG. 8 is a flow diagram that depicts yet another variation of the present method that provides for confirming the identity of a user;

FIG. 9 is a flow diagram that depicts the matter in which a flow directive is received according to various alternative example methods;

FIG. 10 is a flow diagram that depicts one alternative example method that supports hands-free operation of a surgical scrub station;

FIG. 11 is a flow diagram that depicts one variation of the present method wherein a user's identification forms the basis of controlling the temperature of water delivered from a faucet;

FIG. 12 is a flow diagram that depicts one alternative example method wherein an indication is provided to a user once the user has adequately satisfied a surgical scrub profile;

FIGS. 13 and 14 are flow diagrams that depict various alternative example methods for controlling flow rate based upon the temperature of incoming water;

FIG. 15 is a flow diagram that depicts one variation of the present method wherein water is augmented with an antimicrobial agent;

FIGS. 16 and 17 are flow diagrams that depict various alternative methods for controlling the temperature of the mixed water to be delivered to a user;

FIG. 18 is a block diagram of one example embodiment of a water delivery apparatus;

FIG. 19 is a mechanical illustration that also depicts one example embodiment of the water delivery apparatus;

FIG. 20 is a pictorial diagram that illustrates one alternative example embodiment of a control unit;

FIGS. 21A and 21B are pictorial diagrams that illustrate two alternative methods for coupling the mixing unit to the control unit;

FIG. 22 is a pictorial diagram that illustrates one alternative example embodiment of a mixing unit;

FIG. 23A is a block diagram that further details several alternative example embodiments of a control unit;

FIG. 23B is a pictorial diagram that illustrates various user expressions as they correlate to particular types of directives that the gesture sensor is capable of perceiving;

FIG. 24A is a block diagram that depicts several alternative example embodiments of a flow rate controller, which is included in the mixing unit;

FIG. 24B is a block diagram of one example alternative embodiment of the temperature/flow rate controller;

FIG. 25 is a block diagram that depicts several alternative example embodiments of a compliance unit; and

FIGS. 26 and 27 collectively constitute a block diagram of one alternative example embodiment of a flow rate controller that is based on a microprocessor.

### DETAILED DESCRIPTION

Today, surgical infection control is made more effective using new methods for controlling the application of water and/or antimicrobial agents during surgical scrub procedures. These new methods, along with variations thereof, are augmented by application of handwashing profiles. Also, other variations of the present method provide for confirmation that surgeons and other surgical staff have properly washed their hands and/or have properly applied antimicrobial agents before putting on gloves and entering the surgical theater.

In the interest of clarity, several example alternative methods are described in plain language. Such plain language descriptions of the various steps included in a particular method allow for easier comprehension and a more fluid description of a claimed method and its application. Accordingly, specific method steps are identified by the term “step” followed by a numeric reference to a flow diagram presented in the figures, e.g. (step 5). All such method “steps” are intended to be included in an open-ended enumeration of steps included in a particular claimed method. For example, the phrase “according to this example method, the item is processed using A” is to be given the meaning of “the present method includes step A, which is used to process the item”. All variations of such natural language descriptions of method steps are to be afforded this same open-ended enumeration of a step included in a particular claimed method.

Unless specifically taught to the contrary, method steps are interchangeable and specific sequences may be varied according to various alternatives contemplated. Accordingly, the claims are to be construed within such structure. Further, unless specifically taught to the contrary, method steps that include the phrase “ . . . comprises at least one or more of A, B, and/or C . . . ” means that the method step is to include every combination and permutation of the enumerated elements such as “only A”, “only B”, “only C”, “A and B, but not C”, “B and C, but not A”, “A and C, but not B”, and “A and B and C”. This same claim structure is also intended to be open-ended and any such combination of the enumerated elements together with a non-enumerated element, e.g. “A and D, but not B and not C”, is to fall within the scope of the claim. Given the open-ended intent of this claim language, the addition of a second element, including an additional of an enumerated element such as “2 of A”, is to be included in the scope of such claim. This same intended claim structure is also applicable to apparatus and system claims.

In many cases, description of various alternative example methods is augmented with illustrative use cases. Description of how a method is applied in a particular illustrative use case is intended to clarify how a particular method relates to physical implementations thereof. Such illustrative use cases are not intended to limit the scope of the claims appended hereto.

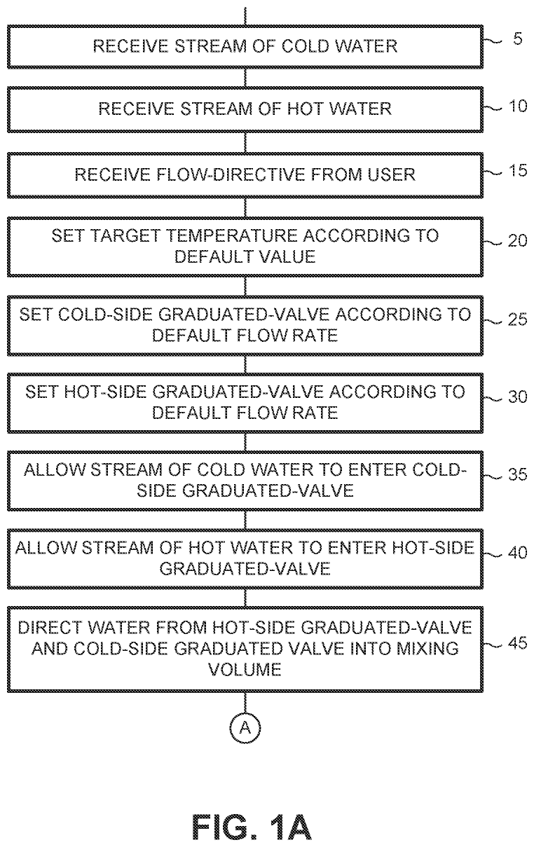

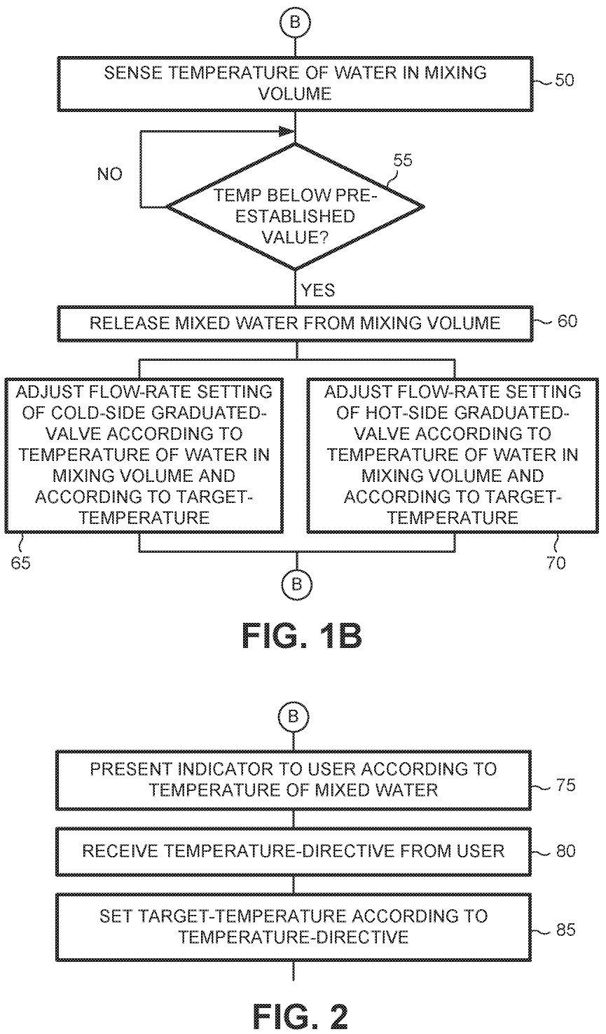

FIGS. 1A and 1B constitute a flow diagram that depicts one example method for delivering temperature controlled water. It should be appreciated that, at the crux of the matter, the temperature of water to be applied during surgical scrub is controlled in order to improve the efficacy of infection control prior to surgery. According to this example method, a stream of cold water is received (step 5) in a first step included in this method. A stream of hot water is also received (step 10) in another step included in this method.

In this example method, a flow-directive is received from a user (step 15). It should be appreciated that, according to this included step of present method, a user is typically a surgeon and/or other surgical staff preparing to enter a surgical theater. A target temperature value is established according to a default value (step 20). It should be appreciated that, according to this present example method, this included step is accomplished sometime before delivering a flow of water to the user. In other words, establishing the target temperature, according to one variation of the present method, is established even before receiving a flow-directive from a user.

This example method includes a step for controlling a cold-side graduated valve according to a default flow rate (step 25). An additional step is included for controlling a hot-side graduated valve according to the default flow rate (step 30). Additional included steps provide for allowing the stream of cold water to enter the cold-side graduated valve (step 35) and also allowing the stream of hot water to enter the hot-side graduated valve (step 40). The water from the hot-side graduated valve and the water from the cold-side graduated valve are then directed into a mixing volume (step 45) in an additional included step of the present method.

This example method includes a step for sensing the temperature of the water in the mixing volume (step 50). When, according to an additional included step, the temperature of the water in the mixing volume is below a pre-established value (step 55), water from the mixing volume is released and directed to the user.

Once water is flowing, this example method includes a step for adjusting the flow rate setting of at least one or more of the cold-side graduated-valve (step 65) and/or adjusting the flow rate of the hot-side graduated-valve (step 70). It should be appreciated that, according to this example method, these additional steps are accomplished by adjusting the flow-rate settings according to a target-temperature. It should also be appreciated that, according to this example method, the flow-rate settings are also adjusted in order to maintain a flow rate according to the default flow rate as established as heretofore described.

FIG. 2 is a flow diagram that depicts one variation of the present method wherein a user is allowed to provide a temperature-directive. According to this variation of the present method, a step is included for presenting an indicator to a user according to the temperature of the mixed water (step 75). It should be appreciated that, according to yet another variation of the present method, the step for presenting an indicator according to the temperature of the mixed water is an optional step. An additional included step provides for receiving a temperature-directive from the user (step 80). When a temperature directive is received from the user, an additional included step of this variation of the present method provides for setting the target-temperature accord project very well be in Orlando next week ing to the received temperature-directive.

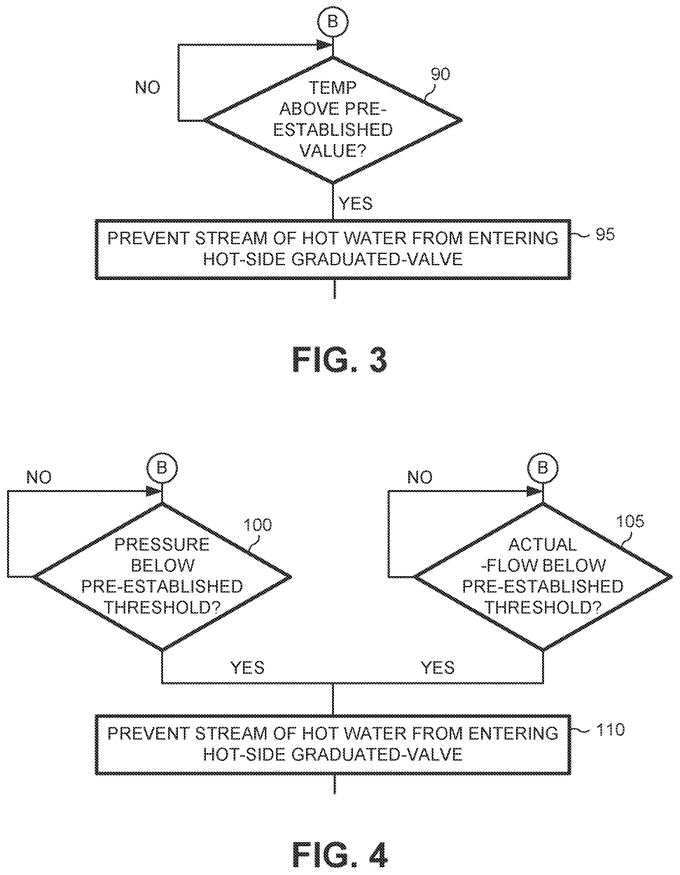

FIG. 3 is a flow diagram one alternative example method that includes steps for a temperature safety cut-off for an incoming hot water stream. In this example variation of the present method, the temperature of the mixed water is measured and compared, in one included step, against a pre-established value (step 90). It should be appreciated that, in the event that the temperature of the mixed water is greater than the pre-established value, which in this variation of the present method constitutes a maximum temperature value, the stream of hot water, and yet another included step, is prevented from entering the hot-side graduated-valve (step 95). It should likewise be appreciated that, according to this variation of the present method, the stream of hot water is rapidly clamped in order to further assure safety and prevent scalding water from reaching a user. According to one illustrative use case, this variation of the present method is applied in an apparatus wherein a rapid action solenoid valve is used to gate off the incoming hot water stream from the hot-side graduated-valve.

FIG. 4 is a flow diagram that depicts one variation of the present method wherein characteristics of an incoming stream of cold water are monitored in order to mitigate the potential for scalding water from reaching a user. According to one variation of the present method, the pressure of an incoming stream of cold water is measured. When, according to one included step, the pressure of the incoming stream of cold water falls below a pre-established threshold (step 100), hot water is prevented from entering the hot-side graduated-valve (step 110). In yet another variation of the present method, the flowrate of an incoming stream of cold water is measured. In the event that the flowrate falls below a pre-established threshold (included step 105), hot water is, according to another included step, prevented from entering the hot-side graduated-valve (step 110).

It should be appreciated that, according to one illustrative use case, a pressure sensor is disposed so as to allow a controller to measure the pressure of an incoming stream of cold water. Accordingly, the controller, by comparison against a pre-established threshold value, actuates a rapid action solenoid valve in order to gate off the incoming hot water stream from the hot-side graduated-valve. According to yet another illustrative use case, a flow sensor is disposed so as to allow controller to measure the pressure of an incoming stream of cold water. In this illustrative use case, a controller compares a measured flow rate to a pre-established threshold value and will actuate a rapid-action solenoid valve in order to gate off hot water from the hot-side graduated-valve.

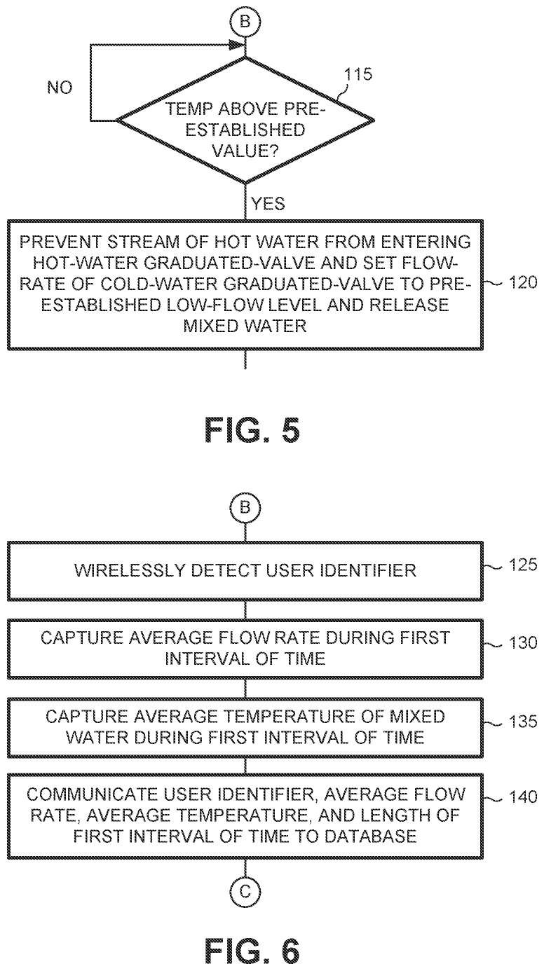

FIG. 5 is a flow diagram that illustrates yet another variation of the present method for avoiding application of scalding water based on the temperature of the mixed water. Accordingly, this variation of the present method includes a step for comparing the temperature of the mixed water against a pre-established value (step 115) and an additional included step for preventing the stream of hot water from entering the hot-water graduate-valve (step 120). This variation of the present method also includes substantially contemporaneous steps for setting the flowrate of the cold-water graduated-valve to a pre-established low-flow level and releasing the mixed water (step 120).

It should be appreciated that, according to one illustrative use case, a temperature sensor is disposed so as to allow a controller to measure the temperature of the mixed water before it is provided to a user. In the event that, according to this illustrative use case, the temperature of the mixed water is greater than a pre-established maximum temperature level, the stream of hot water is prevented from entering the hot-water graduate-valve. In this illustrative use case, this is also accomplished by means of a rapid action solenoid valve which is used to cut off water from entering the hot-water graduate-valve. It should also be appreciated that, according to this illustrative use case, the rapid action solenoid valve is actuated by a controller that includes a comparator four determining when the temperature of the mixed water is greater than the pre-established maximum temperature level.

FIG. 6 is a flow diagram that depicts an alternative variation of the present method wherein characteristics of a surgical scrub cycle are recorded for compliance confirmation purposes. As heretofore described, it is important to make sure that a particular surgeon, and/or surgical staff, properly wash their hands before entering into the surgical theater. Accordingly, this variation of the present method provides an included step for wirelessly detecting a user identifier (step 125).

According to various illustrative use cases, this is accomplished by means of a radio frequency identification tag, commonly referred to as “RFID”. As such, an RFID device associated with a particular user, for example by means of an RFID chip included in an identity card or an identity badge, is wirelessly scanned in order to obtain a user identifier. Various forms of user identifiers are contemplated, for example an employee number, and any example forms of a user identifier here in described are intended to aid in comprehension of the present method and are not intended to limit the scope of the claims appended hereto.

As the user begins to wash their hands, an average flow rate is captured for a first interval of time (included step 130). An average temperature of the mixed water during the first interval of time is also captured in an additional included step of this variation of the present method (step 135). The length of the first interval of time, the average flow rate within that interval of time, and the average temperature of the mixed water during the first interval of time are then communicated along with the user identifier to a database (step 140) in yet an additional included step of this variation of the present method.

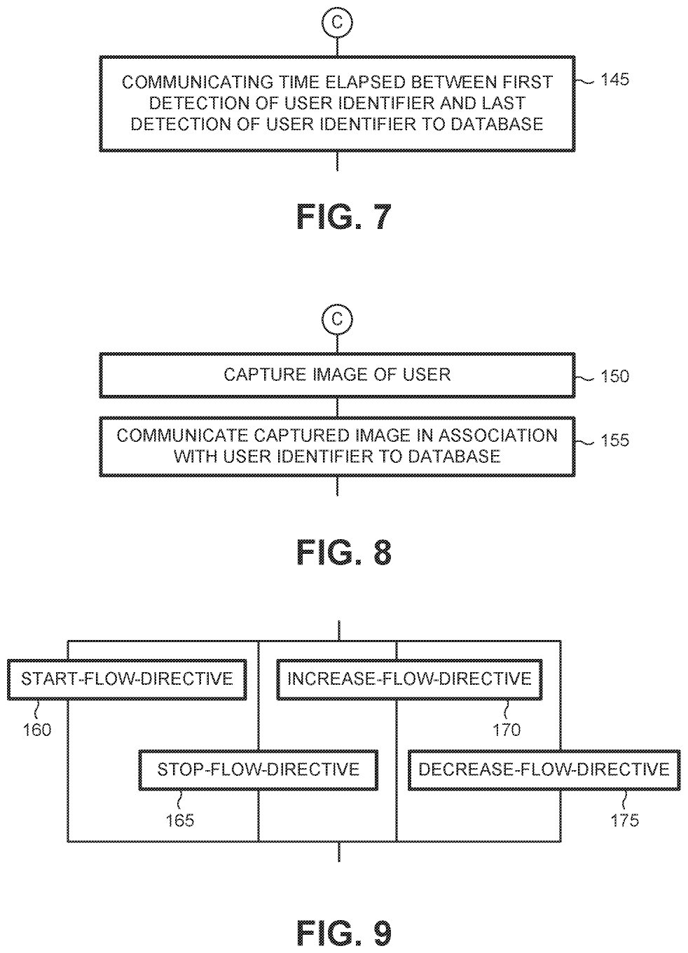

FIG. 7 is a flow diagram that depicts a variation of the present method wherein the amount of time spent by a user washing their hands is recorded. It should be appreciated that, to further bolster compliance with surgical scrub protocols, this variation of the present method further includes a step for communicating the amount of time that a user actually spent washing their hands. In this variation of the present method, the included step provides for communicating the time that has elapsed between the first time a user identifier has been detected and the last time the user identifier has been detected in order to determine an elapsed time of usage. This elapsed time is then communicated along with the user identifier to the database (step 145).

FIG. 8 is a flow diagram that depicts yet another variation of the present method that provides for confirming the identity of a user. This variation of the present method includes a step for capturing an image of the user (step 150) and then communicating the captured image in association with the user identifier to a database (step 155). It should be appreciated that, according to various illustrative use cases, a camera included in a washing station is used to capture an image when a user begins washing their hands. In yet another variation of the present method, an included step provides that video is captured in order to fully record the process by which a user washes their hands in preparation for surgery. As such, this variation of the present method further includes a step for communicating the video along with a user identifier to a database.

FIG. 9 is a flow diagram that depicts the matter in which a flow directive is received according to various alternative example methods. It should be appreciated that, according to one variation of the present method, a step is provided for receiving a flow directive that comprises a start-flow-directive (step 160). It should be appreciated that, according to one illustrative use case, a user may approach a surgical scrub station and indicate to a controller that water should begin flowing from a faucet, thereby enabling the user to wash their hands using said flowing water.

In yet another alternative example method, a step is provided for receiving a stop-flow-directive (step 165). It should likewise be appreciated that, once a user has completed their surgical scrub activity, that user is likely to indicate that waterflow should be discontinued.

In yet another alternative example method, a step is provided for receiving a flow directive that comprises an increase-flow-directive (step 170). It should likewise be appreciated that, as a user is washing their hands, that user may wish to increase the flow of water from a faucet.

And in yet another example method, a step is provided for receiving a flow directive that comprises a decrease-flow-directive (step 175). Just as a user may wish to increase the flow of water during a surgical scrub cycle, that user may wish to decrease the flow of water.

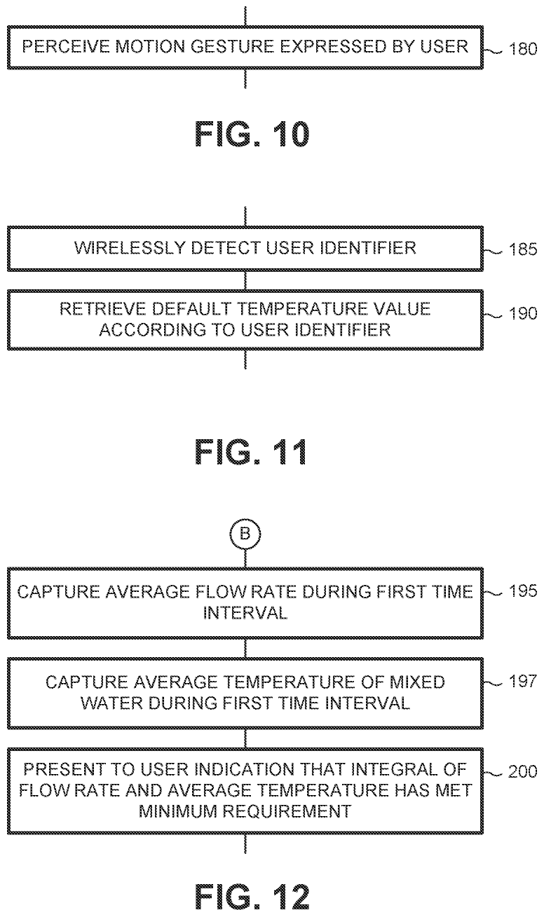

FIG. 10 is a flow diagram that depicts one alternative example method that supports hands-free operation of a surgical scrub station. It should likewise be appreciated that one factor that contributes to re-contamination of a user's hands is the fact that the user must turn off a water faucet after the user has completed a surgical scrub process. This, of course, provides an opportunity for microbial matter on the faucet, for example a faucet control lever, to find its way back onto the surgeon's hands. Although some prior art relies on foot operated control of a faucet, such operated controlled faucets are costly and difficult to operate.

According to this alternative example variation of the present method, a step is provided for receiving a flow directive by perceiving a motion gesture expressed by a user (step 180). It should be appreciated that, according to various illustrative use cases, this alternative example method is implemented in a system wherein an air-gesture sensor is integrated with a flow controller. It should be appreciated that such an error-gesture sensor constantly monitors a volume of space immediately proximate to the sensor and detects when a user's hand is moved into the volume of space and the manner in which the hand is moved within that volume of space.

FIG. 11 is a flow diagram that depicts one variation of the present method wherein a user's identification forms the basis of controlling the temperature of water delivered from a faucet. According to this alternative example method, an included step provides for wirelessly detecting a user identifier (step 185) and then another included step for retrieving a default temperature value according to the user identifier (step 190).

According to one illustrative use case, this variation of the present method is applied in a controller that includes a wireless device for retrieving a user identifier from an RFID chip. Accordingly, the user identifier is used by the controller to retrieve a default temperature value, which the controller then uses to set an initial temperature for delivery of water to the faucet. It should be appreciated that the default temperature value is retrieved from various sources, as according to yet alternative example methods hereto. For example, a controller, according to one embodiment, includes a memory for storing default temperature values based upon user identifier. In yet another embodiment, the controller includes an interface for communicating with a database. In this alternative illustrative use case, the user identifier is communicated by way of the interface to the database and the database responds with a default temperature value. There are numerous ways for maintaining a default temperature value in association with a particular user identifier and the examples set forth herein are not intended to limit the scope of the claims appended hereto.

FIG. 12 is a flow diagram that depicts one alternative example method wherein an indication is provided to a user once the user has adequately satisfied a surgical scrub profile. In this alternative example method, a step is provided for capturing an average flow rate during a first interval of time (step 195). An additional step is provided for capturing an average temperature of the mixed water provided to user during the same first interval of time (step 197). On a continuous basis, this alternative example method provides for calculating an integral according to the average flow rate and the average temperature of the mixed water over the first interval of time. When the calculated integral reaches a pre-establish minimum value, an additional included step provides for giving an indication to the user (step 200) that the user has met the requirements for a particular surgical scrub profile.

It should likewise be appreciated that, according this alternative example method, a surgeon, or other surgical staff no longer need to guess as to whether or not they have fulfilled a minimum requirement as to washing of their hands and elbows. Accordingly, the pre-establish minimum, according to various alternative example methods, is based upon at least one or more of a particular profile established by a hospital or institution, a particular profile established by a regulatory body such as the United States FDA, and/or a particular profile established by an international agency such as the WHO. It should likewise be appreciated that such profiles, according to other alternative example methods, are selected according to the type of surgical procedure that is contemplated by the surgeon and/or surgical staff.

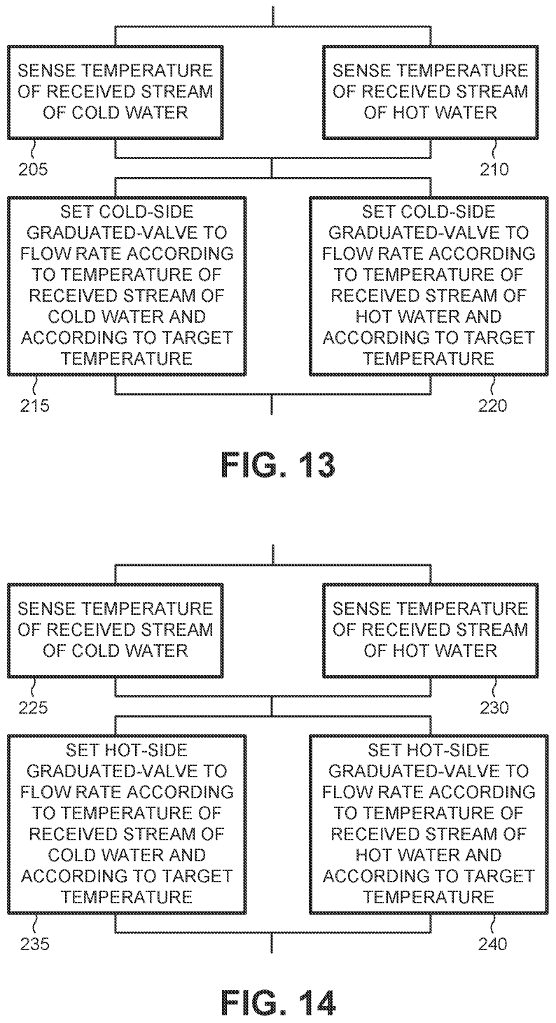

FIGS. 13 and 14 are flow diagrams that depict various alternative example methods for controlling flow rate based upon the temperature of incoming water. It should be appreciated that, according to the present method, one aspect of controlling flow rate is to control flow rate in order to achieve a particular mixed water temperature. Accordingly, a first variation of the present method includes a step for sensing the temperature of a received stream of cold water (step 205). An additional variation of the present method includes a step for sensing the temperature of a received stream of hot water (step 210).

Given that a controller, according to one illustrative use case, senses the temperature of at least one or more of a received stream of cold water and a received stream of hot water, the controller is able to control mixing of cold water and hot water in order to achieve a particular target temperature for the mixed water. As such, one variation of the present method includes a step for setting the flow rate of a cold-side graduated-valve according to the temperature of the received stream of cold water and according to a target temperature for the mixed water (step 215). And in yet another variation of the present method, a step is included for setting the flow rate of a cold-side graduated-valve according to the temperature of the received stream of hot water and according to the target temperature (step 220).

It should likewise be appreciated that, according to various illustrative use cases, these variations of the present method are applied in a manner such that both the temperature of the received stream of cold water and the temperature of the received stream of hot water are both utilized collectively in order to set the flow rates of those respective streams of water so as to achieve a particular target temperature for the mixed water to be delivered to a user. And, according to yet another illustrative use case, the these variations of the present method are further augmented wherein the flow rates for the stream of cold water and the stream of hot water are also adjusted not only in accordance with a target temperature for the mixed water, but also according to a target flow rate at which the mixed water is to be delivered to a user for the purpose of washing hands in preparation for surgery.

In order to fully appreciate these variations of the present method, is important to understand that according to yet another variation of the present method, the temperature of the received stream of cold water is sensed (step 225) in one included step and the temperature of the received stream of hot water is sensed (step 230) in an additional included step. As such, one variation of the present method provides a step for setting a hot-side graduated-valve to a flow rate according to the temperature of the received stream of cold water (step 235). And in yet another variation of the present method, a step is provided for setting the hot-side graduated-valve to a flow rate according to the temperature of the received stream of hot water (step 240).

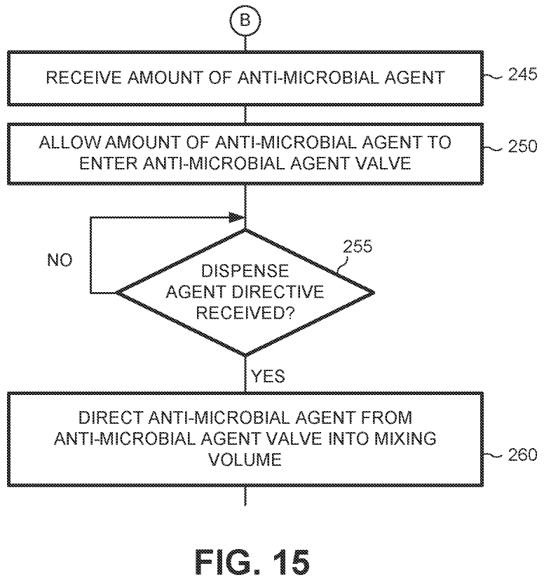

FIG. 15 is a flow diagram that depicts one variation of the present method wherein water is augmented with an antimicrobial agent. It should likewise be appreciated that, according to one variation of the present method, it is desirable to introduce an antimicrobial agent into the stream of mixed water prior to delivering the mixed water to a user for the purpose of engaging in a surgical scrub activity. Accordingly, one variation of the present method includes a step for receiving an amount of anti-microbial agent (step 245). It should likewise be appreciated that, according to one illustrative use case, an antimicrobial agent is received into a reservoir proximate to a mixing volume which is used to mix hot water and cold water before directing the mixed water to a user.

This variation of the present method also includes a step for directing the antimicrobial agent to an antimicrobial agent valve (step 250). According to one illustrative use case, a path is provided from a reservoir for storing the antimicrobial agent to a valve. In this illustrative use case, the valve is used to selectively allow an amount of antimicrobial agent to enter the mixing volume.

Also according to this variation of the present method, a step is included for receiving a dispense agent directive (step 255). When a dispense agent directive is received (step 255), this variation of the present method includes a step for directing the antimicrobial agent from the antimicrobial agent valve into the mixing volume (step 260). It should be appreciated that, according to one illustrative use case, this step of this variation of the present method is applied by a controller by actuating the antimicrobial agent valve in order to allow a volume of antimicrobial agent to enter the mixing volume.

It should be appreciated that, according to one illustrative use case, the directive to dispense an antimicrobial agent is received from a user, for example by means of sensing an air-gesture expressed by said user. According yet another illustrative use case, a directive to dispense an antimicrobial agent is received from a controller as a controller follows a particular surgical scrub profile. For example, a particular surgical scrub profile, according to this illustrative use case, includes a hand washing phase followed by a sanitization phase. During said sanitization phase, the controller releases a volume of antimicrobial agent into a mixing volume by actuating a valve disposed in between a reservoir for the antimicrobial agent and the mixing volume.

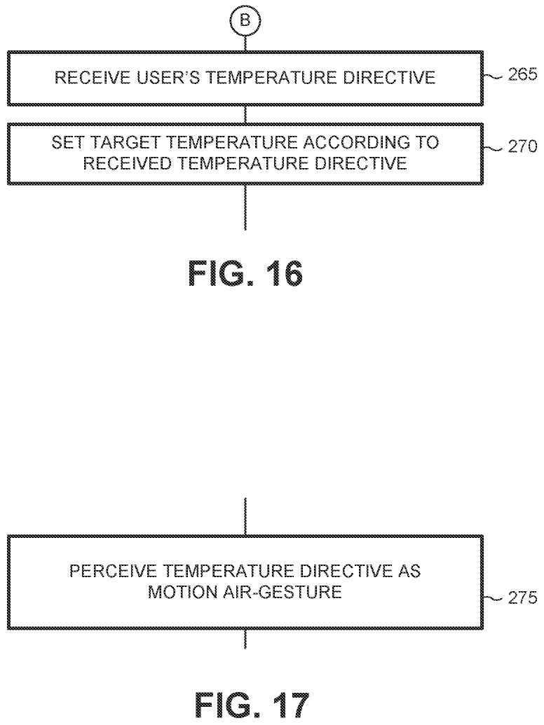

FIGS. 16 and 17 are flow diagrams that depict various alternative methods for controlling the temperature of the mixed water to be delivered to a user. It should be appreciated that, as heretofore described, various example methods presented herein rely upon a default temperature value, which is used to establish an initial temperature for the mixed water. However, a user may wish to adjust the temperature of the water. In such cases, one alternative example method provides a step for receiving a temperature directive from a user (step 265). Accordingly, this variation of the present method provides a step for setting the target temperature according to the received temperature directive (step 270).

In order to further mitigate the potential for re-contamination, one alternative example method provides a step for receiving a temperature directive from a user in the form of an air-gesture (step 275). It should likewise be appreciated that, according to one illustrative use case, a controller perceives hand motions expressed by a user in a particular volume of free space. The controller perceives these hand motions and interprets such motions directives expressed by the user.

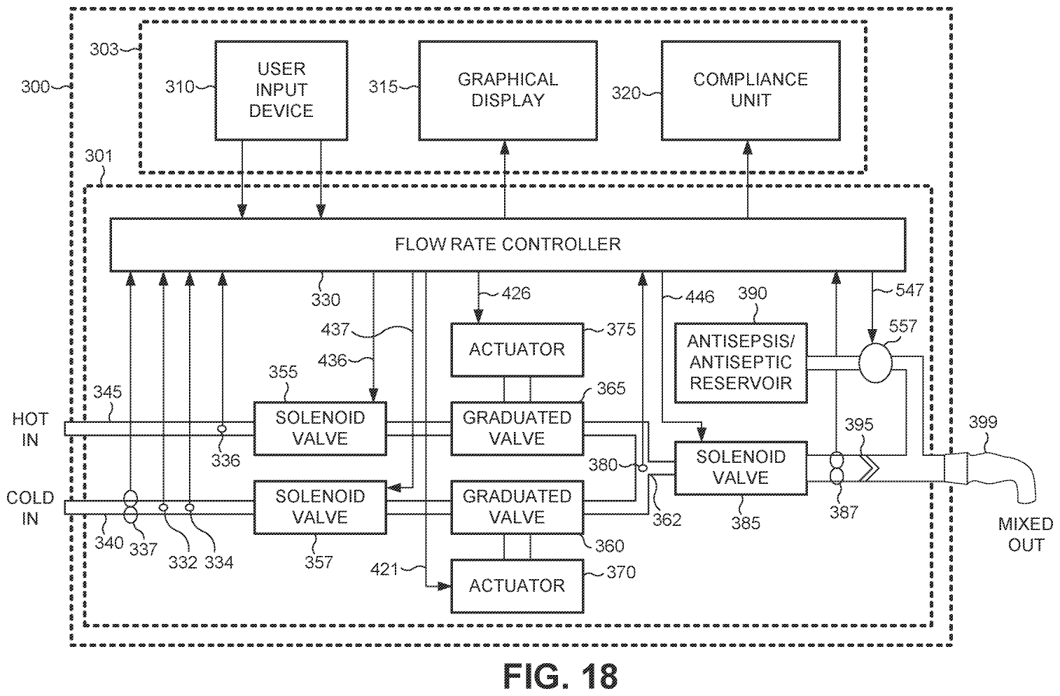

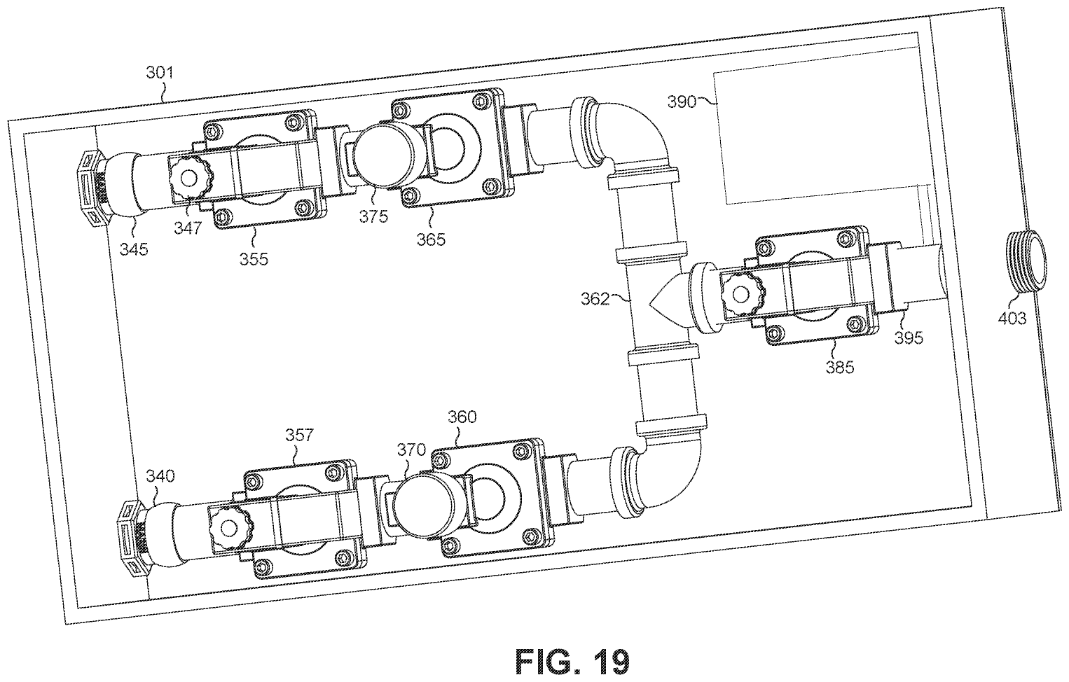

FIG. 18 is a block diagram of one example embodiment of a water delivery apparatus. FIG. 19 is a mechanical illustration that also depicts one example embodiment of the water delivery apparatus. According to this example embodiment, the water delivery apparatus 300 comprises a cold-side solenoid valve 357. In this example embodiment, a cold-side input port 340 is also included for receiving cold water. The input of the cold-side solenoid valve 357 is coupled to the cold-side input port 340.

This example embodiment also includes a cold-side graduated valve 360, which includes an output and input. The input of the cold-side graduated valve 360 is connected to the output of the cold-side solenoid valve 357. The cold-side graduated valve 360 is mechanically coupled to a cold-side actuator 370. In operation, the cold-side actuator 370 adjusts the amount of water that is allowed to flow through the cold-side graduated valve 360.

According to this example embodiment, the water delivery apparatus also comprises a hot-side solenoid valve 355. In this example embodiment, a hot-side input port 345 is also included for receiving hot water. The input of the hot-side solenoid valve 355 is coupled to the hot-side input port 345.

This example embodiment also includes a hot-side graduated valve 365, which includes an output and input. The input of the hot-side graduated valve 365 is connected to the output of the hot-side solenoid valve 355. The hot-side graduated valve 365 is mechanically coupled to a hot-side actuator 375. In operation, the hot-side actuator 375 adjusts the amount of water that is allowed to flow through the hot-side graduated valve 365.

FIG. 18 also illustrates that, according to this example embodiment, the water delivery apparatus 300 also includes a mixing volume 362. The mixing volume 362 receives hot water from the hot-side graduated valve 365 and it receives cold water from the cold-side graduated valve 360. Accordingly, the mixing volume 362 is coupled to the outputs of the hot-side graduated valve 365 and the cold-side graduated valve 360.

As water from the two graduated valve enters the mixing volume 362, it is mixed in order to develop water at a particular temperature. Accordingly, this example embodiment further includes a temperature sensor 380 disposed to sense the temperature of the water in the mixing volume 362. This temperature sensor is referred to as the output-temperature-sensor 380. The output-temperature-sensitivity and 80 generates a signal according to the temperature of the water in the mixing volume 362.

This example embodiment further includes a user input device 310. The user input device 310 perceives directives directed at the water delivery apparatus 300 by a user. In one alternative example embodiment, the user input device 310 comprises an error-gesture sensor, which senses air-gestures expressed by a user using the water delivery apparatus 300. In this example embodiment, the user input device 310 generates a flow-directive signal when a flow-directive is received from a user.

In some embodiments, the water delivery apparatus 300 is organized into a mixing unit 301 and a control unit 303. As such, the mixing unit 301, according to some embodiments, includes a flow rate controller 330. As further described infra, the flow rate controller 330, according to some alternative example embodiments, includes a target-temperature register and a flow-rate register. In operation, the flow-rate register maintains a target-flow-rate value, which is adjusted according to flow-directive signals received from the user input device 310. Accordingly, as a user expresses a flow-directive, signals from the user input device 310 cause the value in the flow-rate register to be increased and/or decreased according to the type of flow-directive expressed by a user.

According to this example embodiment, the target-temperature register provides an initial target-temperature value. The flow-rate-controller 330 of this example embodiment generates a cold-side-flow signal and a hot-side-flow signal according to the target-temperature value maintained by the target-temperature register. The four-rate-controller 330 also uses the output-temperature-signal generated by the upper-temperature-sensor 380 in order to adjust the cold-side-flow signal and the hot-side-flow signal in order to minimize the difference between the temperature as is sensed by the output-temperature-sensor 380 and the target-temperature value maintained by the target-temperature register.

In this example embodiment, the flow-rate-controller 330 further uses the target-flow-rate value maintained by the flow-rate-register to further control the cold-side-flow signal and the hot-side-flow signal. These signals are then directed to the cold-side graduated valve actuator 370 and the hot-side graduated valve actuator 375 in order to affect control over output water temperature and flow rate.

FIG. 18 also illustrates that, according to one alternative example embodiment, the water delivery device 300 further includes a temperature-indicator, which is used to present to user an indication according to the output temperature signal generated by the output-temperature-sensor 380. In this example embodiment, when a user expresses a temperature-directive, the user input device 310 generates the corresponding temperature-directive, which is then used to adjust the value maintained in the target-temperature register.

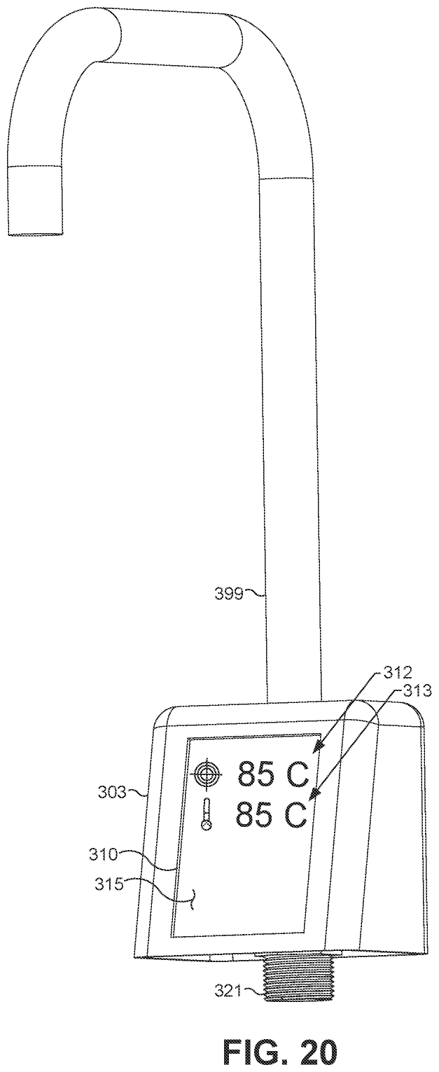

FIG. 20 is a pictorial diagram that illustrates one alternative example embodiment of a control unit. In this alternative example embodiment, the temperature indicator included in the water delivery apparatus includes at least one or more of a target temperature indicator 312 and an actual temperature indicator 313. Accordingly, as a user expresses a temperature directive, the target temperature indicator 312 provides feedback for such temperature directives. At the temperature of the mixed water is adjusted to conform to the target temperature, the output (actual) temperature indicator 313 will reflect the temperature of the water, is sensed by the output-temperature-sensor 380.

FIG. 20 also illustrates that, according to one alternative example embodiment, the water delivery apparatus 300 further includes a faucet 399. As depicted in FIG. 18, the faucet 399 is coupled to the output of an output solenoid which is included in some alternative example embodiments herein described. According to one alternative example embodiment, the faucet 399 includes a national pipe tapered (“NPT”) threaded connector 321, which, according to this alternative example embodiment, is used to attach a plumbing hose, e.g. a faucet to a water supply in residential or industrial applications. FIG. 19 also illustrates that, according to one alternative example embodiment, the water delivery apparatus also includes an NPT threaded connector 403, which, in this alternative example embodiment, is used to attach to a plumbing hose in order to affect connection between the mixing unit 301 and the control unit 303.

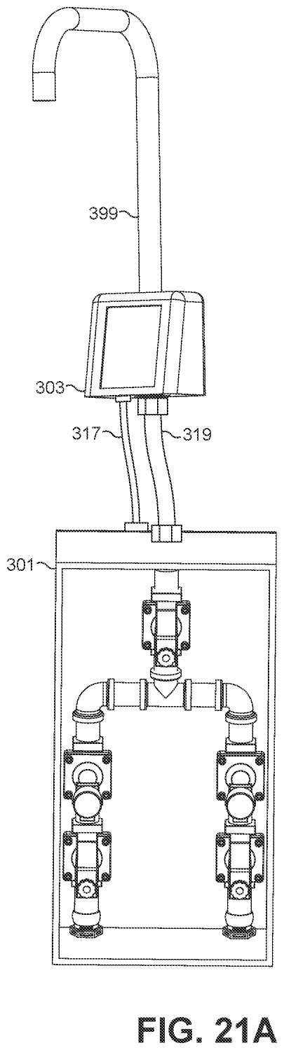

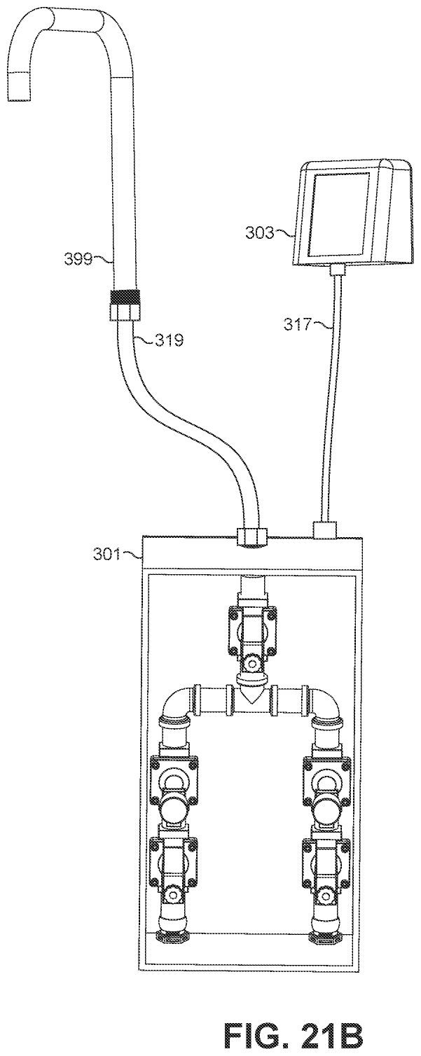

FIGS. 21A and 21B are pictorial diagrams that illustrate two alternative methods for coupling the mixing unit to the control unit. In one alternative example embodiment, the mixing unit 301 includes an electrical connection 317 and a plumbing connection 319. In this alternative example embodiment, the control unit 303 has included there with the faucet 399. Accordingly, the faucet 399 is connected to the mixing unit 301 by way of the first plumbing connection 319. Electronic components included in the control unit 303 are electrically connected to controller electronics included in the mixing unit 301 by way of the electrical connection 317. Additional details of the electrical connection are presented below.

FIG. 21B illustrates that, according to another alternative example embodiment, the control unit 303 is connected to control electronics included in the mixing unit 301 by way of the electrical connection 317. However, this alternative example embodiment recognizes that, in some applications, the faucet 399 is installed disparate from the control unit 303. This, according to some illustrative use cases, provides for use of an existing faucet, for example where an existing faucet is already installed in a washbasin.

This alternative example embodiment also provides for locating the control unit 303 away from the faucet 399. It should be appreciated that, in those applications with the faucet 399 is located too close to the control unit 303, a user may have difficulty interacting with the control unit 303 while there is water flowing from the faucet 399. By supporting this disparate mounting structure, a user is able to freely interact with the control unit 303. Water from the mixing unit 301 is then communicated to the faucet 399 by a plumbing hose 319.

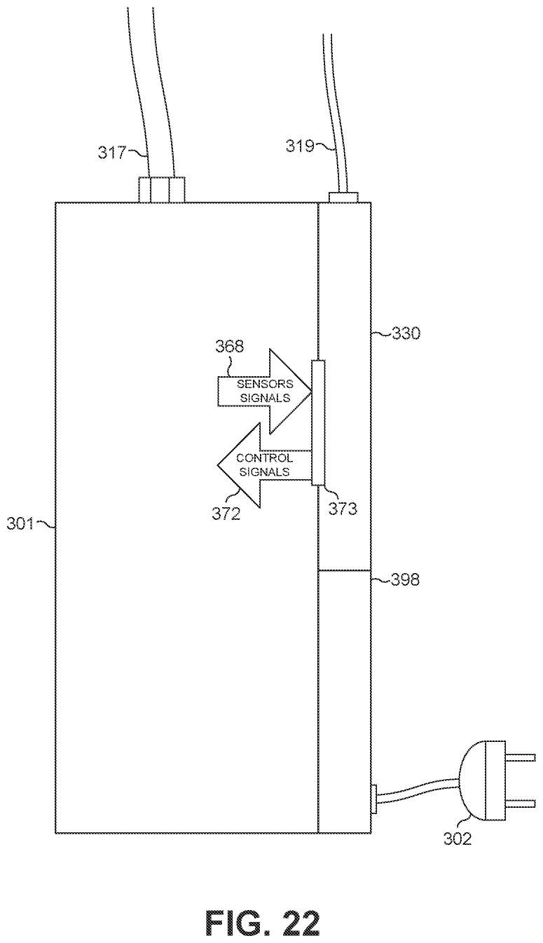

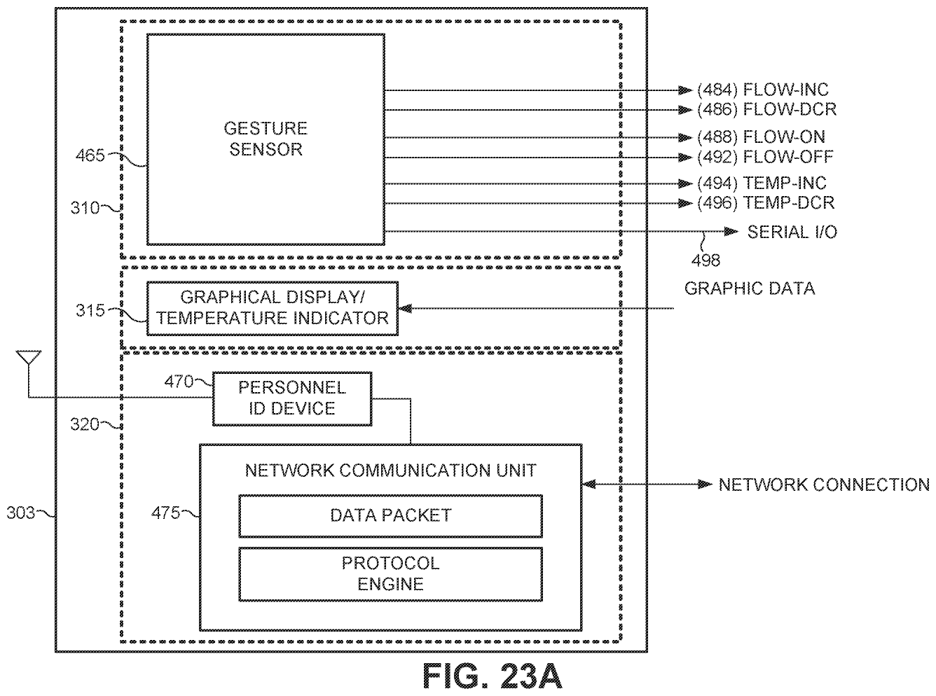

FIG. 22 is a pictorial diagram that illustrates one alternative example embodiment of a mixing unit. In this example embodiment, the mixing unit 301 includes a flow controller 330. The flow controller 330 receives sensor signals 368 from within the mixing unit 301, which is used to enclose the various plumbing elements included in the apparatus herein described. Based upon those sensor signals 368, the flow rate controller 330 develops control signals 372, which are used to control the various actuators included in the mixing unit 301.

This alternative example embodiment also includes a power supply 398, which receives electrical power from the utility output by way of a utility plug 302. The power supply 398 provides electrical power to the flow rate controller 330, and two other sensitive electronic systems included in the apparatus for delivering temperature control water. As heretofore described, the mixing unit 301 provides mixed water by way of a plumbing hose 317 and the flow rate controller 330 is electrically connected to is electrically connected to the control unit 303 by way of an electrical cable 317.

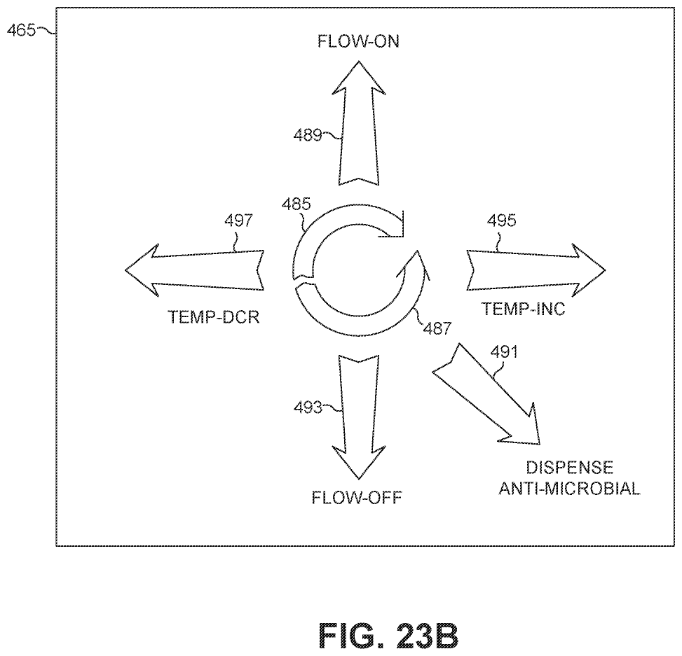

FIG. 23A is a block diagram that further details several alternative example embodiments of a control unit. According to this alternative example embodiment, the control unit 303 includes a user input device 310. In yet another alternative example embodiment, the control unit 303 includes a graphical display 315. And in yet another alternative example embodiment, the control unit 303 includes a compliance unit 320. In this alternative example embodiment, the compliance unit 320 includes a personal identifier device 470 and a network communications unit 475.

In operation, the user input device 310 included in one alternative example embodiment of the control unit 303 includes a gesture sensor 465. The gesture sensor, of this alternative example embodiment, also includes electronic control logic that enables the gesture sensor to perceive various flow directors and/or temperature directives that are or may be expressed by a user. According to one alternative example embodiment, the control logic included in the gesture sensor 465 enables the gesture sensor 465 to perceive at least one or more of a “flow increment” directive 484, a “flow decrement” directive 486, a “flow on” directive 488, a “flow off” directive 492, a “temperature increment” directive 494 and/or a “temperature decrement” directive 496. These individual directives, according to one alternative example embodiment, a communicated from the control unit 303 to the mixing unit 301 by discrete signals as depicted in the figure. However, in one alternative example embodiment, these functional flow and temperature directives are expressed by the gesture sensor 465 by means of a message encoded with a particular type of directive and communicated from the control unit 303 to the mixing unit 301 by way of a serial interface 498.

FIG. 23B is a pictorial diagram that illustrates various user expressions as they correlate to particular types of directives that the gesture sensor is capable of perceiving. It should be appreciated that, according to this alternative example embodiment, the gesture sensor 485 perceives motion is expressed by a user in free space just above a detection surface, which is a surface included in the gesture sensor 485 capable of detecting motion expressed by fingers of a human hand. According to one alternative example embodiment, the gesture sensor 465 perceives an upward wave 489 as a “flow on” directive 488.

According to yet another alternative example embodiment, the gesture sensor 465 perceives a downward wave 493 as a “flow off” directive 493. And in yet another alternative example embodiment, the gesture sensor 465 perceives a rightward wave 495 as a “temperature increment” directive 494. In another alternative example embodiment, the gesture sensor 465 perceives a leftward wave 497 as a “temperature decrement” directive 496. When another alternative example embodiment of the gesture sensor 465 perceives a clockwise rotating finger movement 485, the gesture sensor 465 perceives this as a “flow increment” directive 488. Likewise, the counter clockwise rotating finger movement 487 is perceived by one alternative example embodiment of the gesture sensor 465 as a “flow decrement” directive 486.

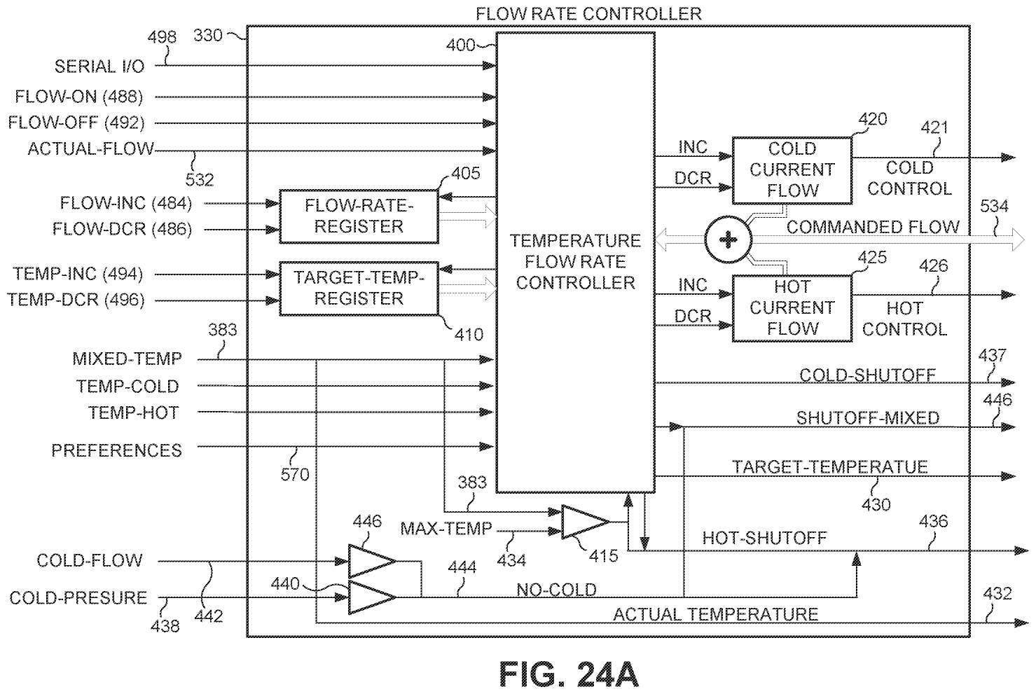

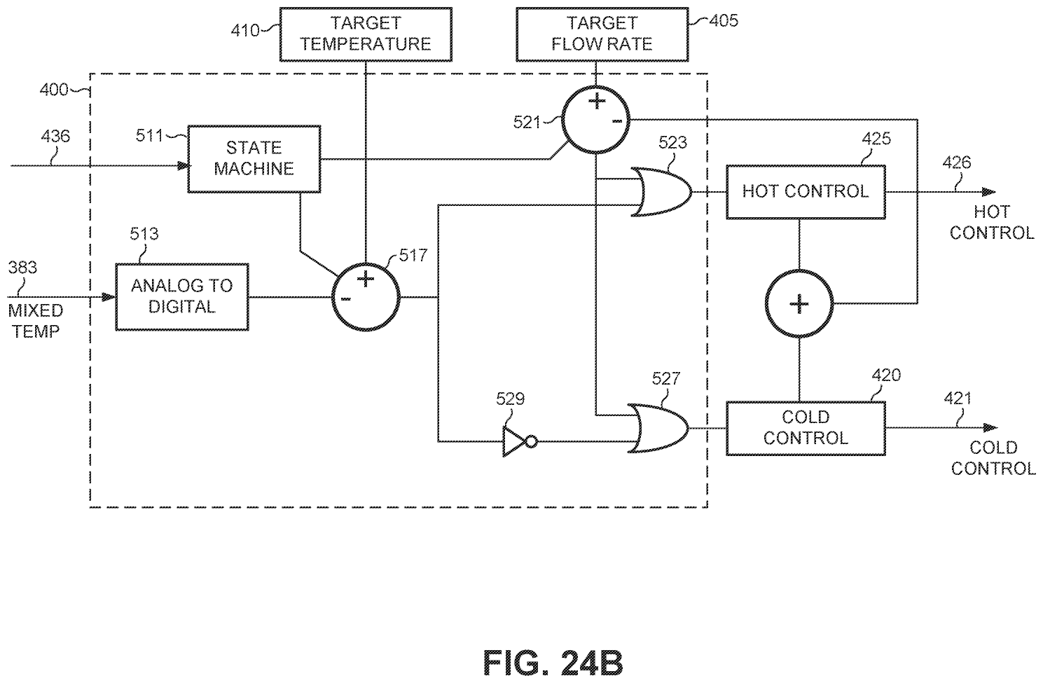

FIG. 24A is a block diagram that depicts several alternative example embodiments of a flow rate controller, which is included in the mixing unit. It should be appreciated that many of the inputs to the flow rate controller 330 are shown as discrete signals. However, one alternative example embodiment of the flow rate controller 330 receives directives from the control unit 303 by way of a serial port 407.

As illustrated, one alternative example embodiment of a flow rate controller 330 includes a flow rate register 405. And in yet another alternative example embodiment, the flow rate controller 330 further comprises a target temperature register 410. In operation, the flow rate controller 330 receives at least one or more of a flow increment directive 484 and/or a flow decrement directive 486 from a user input device 310. As heretofore described, the user input device 310 is included in one alternative example embodiment of a water delivery apparatus. The flow rate register 485 maintains a target flow rate value that is increased when a flow increment directive 484 is received. The target flow rate value maintained in the flow rate register 405 is decreased in response to a received flow decrement directive 486.

As the water delivery apparatus of one alternative example embodiment continues to operate, the flow rate controller 330, which in yet another alternative example embodiment includes a temperature/flow rate controller 400, the temperature/flow rate controller 400 generates a cold-site-flow signal and a half-site-flow signal based on the target temperature value provided by the target temperature register 410, and also according to the target-flow-rate value maintained by the flow rate register 405. In this alternative example embodiment, the temperature/flow rate controller 400 also adjusts the values of at least one or more of the cold-side-flow signal and the hot-site-flow signal according to an output temperature signal, which is also referred to as the mixed temperature signal 383.

In one alternative example embodiment, the temperature/flow rate controller 400 adjusts the cold-side-flow signal and the hot-site-flow signal by issuing increment and decrement signals in order to control a cold-current-flow register 420 and hot-current-flow register 425. The cold-current-flow register 420 generates a cold control signal 421 based on the value currently maintained in said register. The hot-current-flow register 425 generates a hot control signal 426 based on the value currently maintained in the hot-current-flow register 425.

In some alternative example embodiments, the cold control signal 421 and the hot control signal 426 comprise signals to control stepping motor actuators. In these alternative example embodiments, the stepping motor actuators comprise the cold-side-graduated-valve actuator 370 and the hot-side-graduated-valve actuator 375. In yet other alternative example embodiments, the cold control signal 421 and the hot control signal 426 comprise signals that indicate position. In these alternative example embodiments, position signals are directed to stepping motor controllers, one for the cold-side and one for the hot-side. In some other alternative example embodiments, the position signals are directed to servomotor controllers, one for the cold-side and the other for hot-side.

Irrespective of the type of actuator used, the effect is that the hot-side graduated valve 365 and the cold-side graduated valve 360 are commanded by the hot control signal 426 and the cold control signal 421, respectively. Accordingly, the amount of flow allowed through the hot-side graduated valve 365 and the cold-side graduated valve 360 is thus established by the values maintained in the hot-current-flow register 425 and the cold-current-flow register 420, again respectively.

FIGS. 18 and 20 further illustrates that, as heretofore described, one alternative example embodiment of a water delivery apparatus further comprises a temperature indicator 313. According to yet another alternative example embodiment, the water delivery apparatus further includes a target temperature indicator 312. In operation, as a user expresses a temperature increment and/or decrement directive, the target temperature indicator 312 provides feedback to the user as to changes to the target temperature resulting from user expressed temperature increment directives.

Referring to FIG. 24A, the temperature/flow rate controller 400 of this alternative example embodiment receives a value from the target-temperature-register 410, and passes this along as a target-temperature signal 430 the control unit 303, which in one alternative example embodiment includes a target temperature indicator 312. In yet another alternative example embodiment, the target temperature indicator 312 is embodied as a graphical representation, which is presented on the graphical display 315.

In yet another alternative example embodiment, the flow rate controller 330 receives a mixed temperature signal 383 and passes this along to the control unit 303 as an actual temperature signal 432. Accordingly, the control unit 303 presents a mixed temperature indicator 313. It should likewise be appreciated that, according to yet another alternative example embodiment, the target temperature indicator 312 is embodied as a graphical representation that is presented on the graphical display 315.

FIG. 24A also illustrates that, according to one alternative example embodiment, the water delivery apparatus further comprises an emergency temperature comparator 415. In this alternative example embodiment, the emergency temperature comparator 415 includes an input that is connected to the mixed temperature signal 383 and an input that is connected to a pre-established maximum temperature value 434. In operation, when the value of the mixed temperature signal 383 substantially exceeds the maximum temperature value 434, the emergency temperature comparator 415 generates a hot-shutoff signal 436. The hot-shutoff signal 436 is directed to the hot-side solenoid valve 355, which response to the hot-shutoff signal 436 by substantially blocking water flow from the hot water input port 345.

FIG. 24B is a block diagram of one example alternative embodiment of the temperature/flow rate controller. In this alternative example embodiment, a temperature/flow rate controller 400 comprises a state machine 511, an analog to digital converter 513, and a temperature difference unit 517. In this alternative example embodiment, the state machine 511 causes the analog-to-digital converter 513 to sample an analog value of a mixed temperature signal 383. The sampled value is then directed to the temperature difference unit 517. The temperature difference unit 517 generates a different signal according to a value stored in the target temperature register 410 and the sampled value of the mixed temperature signal. The state machine 511 then causes the temperature difference unit 517 to issue one or more pulses according to the difference generated by the temperature difference unit 517. In this alternative example embodiment, the pulses are directed to a hot control register 425 and an inverse of the pulse is directed to the cold control register 420.

In this embodiment, when the mixed temperature 383 is not warm enough, vis-à-vis the value stored in the target temperature register 410, a pulse is directed to the hot control register 425 in order to increase the amount of hot water allowed into the mixing chamber 362. When a pulse is used to increase the value stored in the hot control register 425, a corresponding pulse, as depicted by an inverter symbol 529, is directed to the cold control register 420. This causes the value in the cold control register 422 be decreased according to the pulse. Accordingly, every increase of the hot control register 425 is complemented by a corresponding cold control register 420 decrease. By so doing, the aggregate amount of flow from the mixing volume 362 remains essentially constant.

FIG. 24B also illustrates that, according to one alternative example embodiment, the temperature/flow rate controller 400 further includes a flow rate difference unit 521. The flow rate difference unit 521 receives as feedback the sum of values stored in the hot control register 425 and the cold control register 420. The sum, which represents the aggregate flow rate provided by the hot-side graduated-valve 365 and the cold-side graduated-valve 360, is subtracted from the target flow rate stored in the target flow rate register 405. The state machine 511 of this alternative example embodiment causes the flow difference unit 521 to generate pulses to either increase or decrease the flow, accordingly, either increase or decrease pulses are directed to the hot control register for 25 and the cold control register 420 substantially contemporaneously. As shown in the figure, pulses from the flow difference unit 521 are logically or together with pulses from the temperature difference unit 517, represented by or gates 523 and 527.

It should be appreciated that the temperature/flow rate controller 400 implements the control law described above using standard control law principles, which are well known in the art. For example, according to one alternative example variation, generation of control signals for the hot-side graduated valve 365 and the cold-side graduated-valve 360 is accomplished using the proportional, integral and derivative control principle, often referred to as PID. It should be appreciated that gain and time constants for such a PID control law are typically, and as is the case here developed through empirical techniques well understood in the art of control mechanics

According to one alternative example embodiment, the temperature/flow rate controller 400 responds to the hot-shutoff signal 436 by reducing the value in the cold current flow register 422 a pre-established low-flow value. By so doing, any water still contained in the mixing volume 362 is flushed out from the faucet 399 at a very low rate. This helps to ensure that very little over temperature water reaches a user, thus substantially preventing potential injury.

FIG. 18 also shows that, according to one alternative example embodiment, the water delivery apparatus further comprises a flow sensor disposed to measure the flow rate of the stream of cold water (cold water flow rate sensor 337). The cold water flow rate sensor 337 generates a signal referred to as cold-flow 442, which is received by the temperature flow rate controller 400, see FIG. 24A. This alternative example embodiment also includes a cold flow comparator 446. The cold flow comparator 446 generates a no cold signal 444 when the cold flow signal 442 falls below a pre-established value. The no cold signal 444 is logically combined with the hot shutoff signal 436. Accordingly, the hot shutoff signal 436 is directed to the hot side solenoid valve 355, which responds to the hot shutoff signal 436 by substantially blocking the flow of hot water from the hot water input port 345.

Again referring to FIG. 18, one alternative example embodiment of the water delivery apparatus further includes a pressure sensor 332 disposed to monitor the pressure of the water received by the cold water input port 340. In this alternative example embodiment, as shown in FIG. 24A, the water delivery apparatus further includes a cold pressure comparator 440. The cold pressure comparator 440 is configured to activate the no cold signal 444 when the pressure of the water received by the cold water input port 340 falls below a pre-established threshold. Accordingly, the hot side solenoid valve 355 responds in an analogous manner to the hot shutoff signal 436, which is driven by the no cold signal 444 when the cold pressure comparator 440 detects that the cold water pressure signal 438 received from the pressure sensor 332 indicates that there is insufficient cold water pressure arriving at the cold water input port 340.

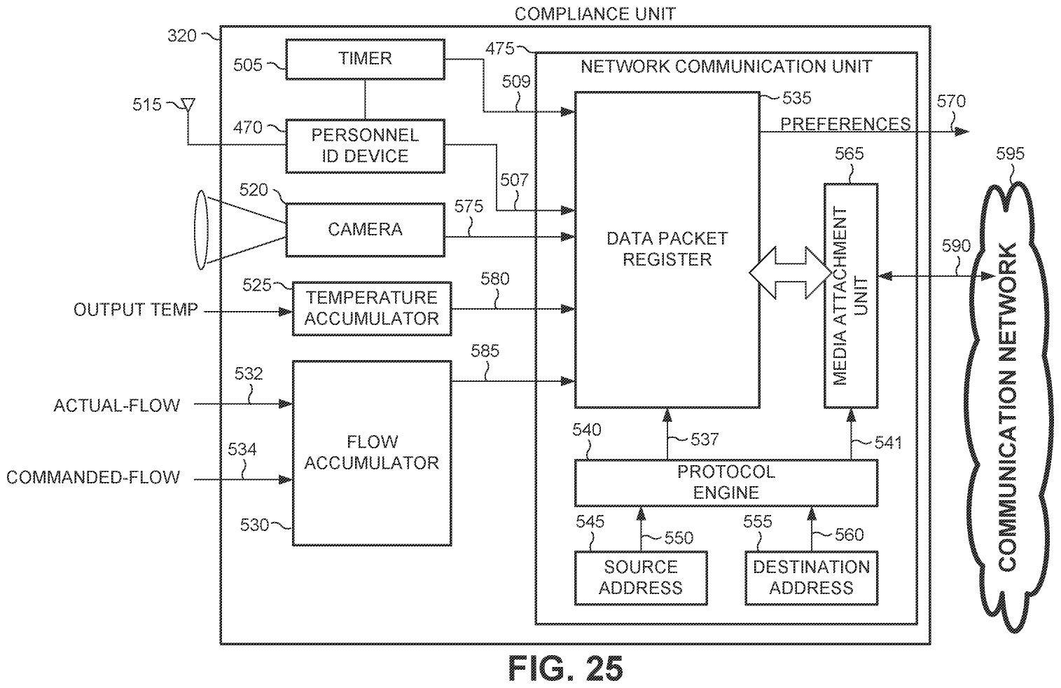

FIG. 25 is a block diagram that depicts several alternative example embodiments of a compliance unit. According to one alternative example embodiment, the compliance unit 320, which according to several example embodiments presented herein is included in the control unit 303, itself comprises a personal identification device 470. According to one alternative example embodiment, the personal identification device 470 includes a wireless identity interrogator, which is configured to perceive a personal identity credential. It should be appreciated to those skilled in the art that various forms of wireless identity credentials are currently in use. In one illustrative use case, the personal identification device 470 is configured to perceive radio frequency identification (“RFID”) identification tags. It should likewise be appreciated that such RFID identification tags are commonly incorporated into personnel badges used in controlling access to facilities such as hospitals.

In operation, the personal identification device 470 perceives a user's personal identity credential, for example an RFID identification tag, in order to identify a particular user. Once a personal identity credential is perceived, the personal identification device 470 generates an identity indicator 507. As a compliance unit 320 of this alternative example embodiment continues to operate, it collects information regarding a surgical scrub procedure engaged in by the identified user. For example, one alternative example embodiment of the compliance unit 320 includes a temperature accumulator 525.

In operation, the temperature accumulator 525 develops an integral according to the output temperature signal (i.e. the mixed temperature signal 383). By so doing, an average temperature is established for a particular scrub procedure engaged in by the identified user. It should be appreciated that the temperature accumulator 525 of one alternative example embodiment also develops a histogram according to the mixed temperature signal 383, thus providing an overall profile of temperature versus time beginning with when a particular user is first identified by way of the personal identification device 470. This, occurs when the personal identification device 470 perceives a personal identity credential associated with a particular user.

This alternative example embodiment of the compliance unit 320 also includes a network communication unit 475. Once a user has completed a surgical scrub procedure, for example when the user's personal identity credential is no longer perceived by the personal identification device 470, the network communication unit 475 communicates the integral and the identity indicator 507 to a computer network. It should be appreciated that the integral comprises at least one or more of a temperature integral 580 and/or a flow rate integral.

In another alternative example embodiment, the compliance unit 320 further includes a timer 505. In this alternative example embodiment, the timer 505 is started as substantially contemporaneously with the time at which a personal identity credential is perceived by the personal identification device 470. The timer 505 is then stopped when the personal identity credential is no longer perceived by the personal identification device 470. In this manner, the timer 505 reflects the amount of time that a particular user engaged in a surgical scrub procedure.

It should likewise be appreciated that, according to various alternative example embodiments, the temperature integral 580 comprises a time-based histogram, which presents the temperature of the output water delivered to a user at various points in time during a surgical scrub procedure. Also, in one alternative example embodiment, the flow integral 585 comprises a time-based histogram which records flow rate versus time in terms of at least one or more of an actual flow rate 532 and a commanded flow rate 534. As such, the flow rate provided to a user during a surgical scrub is communicated to the computer network 595. According to some illustrative use cases, the communications network 595 comprises a wide area network, for example such as the Internet. It should likewise be appreciated that, in yet other illustrative use cases, the communications network 595 comprises a local area network, which is itself provided with a path to a wide area network such as the Internet.

In one illustrative alternative embodiment, the network communication unit 475 comprises a very simple electronic structure that includes a data packet register 535, a media attachment unit 565, a protocol engine 540, a source address register 545, and a destination address register 555. It should be appreciated that, because of the limited purpose of the compliance unit 320, the source address register 545 is programmed with a particular computer network source address corresponding to a particular water delivery apparatus, as herein described. The destination address register 555 is likewise programmed with the network address for a server that interacts with the network communications unit 475 in order to collect compliance data from one or more water delivery apparatus enabled with a compliance unit 320.

In operation, the protocol engine 540 generates control signals 537 to control the capture of data by the data packet register 535. The protocol engine 540 also generates control signals 541 to control the movement of a data packet through a media attachment unit 565 in accordance with a particular network protocol used to communicate 590 information to the communications network 595. It should be appreciated that, according to one alternative example embodiment, the protocol engine 540 is structured and configured to enforce a transfer control protocol/Internet protocol (“TCP/IP”). Those familiar with the art will realize that the TCP/IP protocol relies upon a source address 550, which the protocol engine 540 obtains from the source address register 545 and a destination address 560, which the protocol engine 540 obtains from the destination address register 555. With this information, the TCP/IP protocol is used to communicate a data packet stored in the data packet register 535 to a server by way of the communications network 595.

It should likewise be appreciated that, according to various alternative example embodiments, the protocol engine 540 uses control signals 537 in order to capture at least one or more of an elapsed time value 509 stored in the timer 505, an identity indicator 570 ascertained by the personal identification device 470, the integral of temperature 580 generated by the temperature accumulator 525 and/or a flow integral 585 generated by the flow accumulator 530. In yet another alternative example embodiment, the protocol engine 540 uses the control signals 537 to capture an image 575 from the camera 520, which is included in this alternative example embodiment of a compliance unit 320.

FIG. 25 also illustrates that, according to yet another alternative example embodiment, a compliance unit 320 also includes a camera 520. In operation, the camera 520 captures an image of a user and directs that image 575 to the network communication unit 475 in accordance with control signals 537 generated by the protocol engine 540. All of such data manipulations by the protocol engine 537 are easily implemented by those skilled in the art of electronic design and by those skilled in network communications.

FIG. 25 also illustrates that, according to one alternative example embodiment, the compliance unit 320 provides preferences 570 back to the temperature/flow rate controller 400. In operation, the protocol engine 540, upon receiving a signal from the personal identification device 470 that a personnel identity credential has been perceived, captures the personnel identity credential 507 into the data packet register 535 and communicates this along with a request for preferences to a server by way of the communication network 595. In this alternative example embodiment, the protocol engine 540 directs the preferences 570 from the data packet register once the preferences are received from the server.

On the server side, the server uses the personnel identity credential to retrieve from a database at least one or more of a desired flow rate and/or a desired temperature. Accordingly the preferences 570 include at least one or more of the desired flow rate and/or the desired temperature associated with a particular user. The preferences 570 are received by the temperature/flow rate controller 400. When the temperature/flow rate controller 400 receives the preferences 570, it adjusts at least one or more of the flow rate register 405 and the target temperature register 410 according to the received preferences 570. It should be appreciated that, although the term “preferences” implies a plurality, the term “preferences” is also to be construed in the singular.

Referring to FIG. 18, is important to appreciate that not all of the elements presented in this diagram are essential to proper operation of various alternative embodiments of a water delivery apparatus. For example, one alternative illustrative embodiment includes an input port for receiving cold water 340, a cold-side graduated valve 360, a hot side solenoid valve 355, an input port for receiving hot water 345, and a hot side graduated valve 365. A user input device 310 is also included in this alternative example embodiment. The user input device 310 comprises a device that is capable of perceiving a flow directive from a user and passing this flow directive onto the flow rate controller 330.

Akin to other embodiments herein described, the cold-side graduated-valve 360 includes an input that is connected to the cold-water input port 340 and also includes an output that is connected to a mixing volume 362, which is also included in this alternative example embodiment of a water delivery apparatus. However, this alternative example embodiment does not include a cold-side solenoid valve.

In this alternative example embodiment, the hot water input port 345 is connected to the hot-side solenoid valve 355 and the output of the hot side solenoid valve 355 is connected to the hot-side graduated valve 365. The output of the hot-side graduated valve 365 is also connected to the mixing volume 362. This alternative example embodiment also includes an output temperature sensor 380, which generates an output temperature sensor according to the temperature of the mixed water contained in the mixing volume 362.

In this alternative example embodiment, the flow rate controller 330 generates a cold-side flow control signal 421 and a hot-side flow control signal 426. However, in this embodiment, the flow controller 330 generates these signals according to a target temperature value and according to the output temperature signal generated by the output temperature sensor 380. It should be appreciated that, according to this alternative example embodiment, each graduated valve (365, 370) has associated therewith an actuator (375, 370). These actuators are included in this alternative example embodiment and are controlled by the cold-side flow control signal 421 and the hot-side flow control signal 426, respectively.

FIG. 24A illustrates that other alternative example variations of this alternative embodiment, include a target flow register 405. And in yet other variations of this alternative embodiment, a target temperature register 410 is also included in the water delivery apparatus.

In those variations of this alternative example embodiment that include a target flow register 405, the user input device 310 generates a flow directive when a flow command is received from a user. In this variation of this alternative example embodiment, the flow directives include at least one or more of a flow-on directive 488, a flow-off directive 492, a flow increment directive 484, and/or a flow detriment directive 486. In yet another variation of this alternative example embodiment, the user input device 310 comprises an air gesture sensor, which is capable of perceiving gestures expressed by a user in free space. In this variation of this alternative example embodiment, the target flow register 405 adjusts the value stored therein in accordance with flow directives received from the user input device 310.

The flow rate controller 330 of this variation of this alternative example embodiment adjusts the cold-side flow control signal 421 and the hot-side flow control signal 426 in accordance with the value stored in the flow-rate-register 405 and according to a target temperature value and according to the temperature of the mixed water contained in the mixing volume 362. In this manner, the flow rate controller 330 adjusts the cold-side flow control signal 421 and the hot-side flow control signal 426 in order to obtain a target flow rate and a target temperature.

In those variations of this alternative example embodiment that include a target temperature register 410, the user input device 310 generates temperature directives when a temperature command is received from a user. It should be appreciated that, in this variation of this alternative example embodiment, temperature directives include at least one or more of a temperature increment directive 494 and/or a temperature detriment directive 496. The target temperature register 410 responds to such temperature directives by adjusting the value contained therein according to the received directive. In other words, the target temperature register 410 will decrease the value stored therein when it receives a temperature decrement directive 496 and will increase the value stored therein when it receives a temperature increment directive 494. In this variation of this alternative example embodiment, the flow rate controller 330 will continue to adjust the cold-side flow control signal 421 and the hot-side flow control signal 426 in order to obtain a target temperature based on a temperature value stored in the target temperature register 410 and also according to the mix temperature signal it receives from the mixed temperature sensor 380.

This variation of this alternative example embodiment also includes an emergency temperature comparator 415, which compares the mixed temperature signal 383 to a maximum temperature value 434. Here, when the mixed temperature signal indicates that the temperature of the water contained in the mixing volume 362 is greater than the allowable maximum temperature, the emergency temperature comparator generates a hot shut off signal 436. Akin to other embodiments herein described, the hot side solenoid valve 355 response to the hot shut off signal 436 my substantially blocking water flow from the hot water input port 345.

Also included in this variation of this alternative example embodiment is a pressure sensor 332, which generates a pressure signal according to the pressure of water arriving at the cold input water port 340. This variation of this alternative example embodiment also includes a cold-pressure-comparator 440, which is configured to generate a no cold signal 444 when the pressure signal generated by the pressure sensor 332 indicates that the pressure of water arriving at the cold input port 340 is below a pre-established threshold.

In this variation of this alternative example embodiment, the no cold signal 444 is recast as a shut off mixed water signal 446. The shut off mixed water signal 446 is directed to the mixed water solenoid valve 385, which is included in this variation of this alternative example embodiment. When there is insufficient pressure associated with the water arriving at the cold water input port 340, the mixed water solenoid valve 385 substantially blocks water from exiting the mixing volume 362 when it receives the shut off mixed water signal 446. In this situation, mixed water is not allowed to be delivered to a user from the mixing volume 362, for example by way of an included faucet 399.

This variation of this alternative example embodiment also includes a cold flow sensor 337, which is disposed to measure the flow rate of water as it moves into the system from the cold water input port 340. In this variation of this alternative example embodiment, the cold flow signal 442 is received by a cold flow comparator 446, which generates the no cold signal 444 when the cold flow signal 442 indicates that the flow rate of cold water received at the cold water input port 340 is below a minimum pre-established threshold. In this variation of this alternative example embodiment, the no cold signal 444 is also recast as the shut off mixed signal 446 which, when received by the mixed water solenoid valve 385, causes the mixed water solenoid valve to substantially prevent water from exiting the mixing volume. As a result, water from the mixing volume is not allowed to be directed to a user, for example by way of an included faucet 399.

FIG. 18 also depicts yet another alternative example embodiment of a water delivery apparatus. This alternative example embodiment of a water delivery apparatus comprises an input port for receiving cold water 340, a cold-side graduated valve 360, an input port for receiving hot water 345, a hot side graduated valve 365, and a mixing volume 362.

In this alternative example embodiment, the input port for receiving cold water 340 is connected to the input of the cold-side graduated valve 360. The input port for receiving hot water 345 is likewise connected to the input of the hot-side graduated valve 365. The mixing volume 362 includes a cold side input that is connected to the output of the cold-side graduated valve 360 and a hot side input that is connected to the output of the hot side graduated valve 365. The mixing volume 362 also includes an output, which is used to deliver water to a user at a particular temperature.

This alternative example embodiment further comprises an output temperature sensor 380, which is disposed to measure the temperature of water inside the mixing volume 362. This alternative example embodiment also includes a flow rate controller 330. The flow rate controller 330, in this alternative example embodiment, generates a cold side flow signal 421 and a hot side control signal 426. The flow rate controller 330 generates these signals according to a target temperature value and according to the output temperature signal (i.e. the mixed temperature signal 383).

FIGS. 18 and 19 also show that one variation of this alternative example embodiment further includes an anti-microbial agent reservoir 390. In this variation of this alternative example embodiment, the antimicrobial agent reservoir 309 is used to receive a quantity of anti-microbial agent. In this variation of this alternative example embodiment, the user input device 310 is further configured to perceive a “dispense agent” directive. When the flow rate controller 330 receives the dispense agent directive, it activates a dispense agent signal 547. This variation of this alternative example embodiment further includes a dispense agent valve 557, which, in response to the dispense agent signal 547, allows a quantity of antimicrobial agent to be injected into the output water path. In yet another variation of this alternative example embodiment, an anti-siphon valve 395 is introduced between the mixed water solenoid valve 385 and the output faucet 399 so that an antimicrobial agent is not allowed to propagate back into the water supply.

According to one variation of this alternative example embodiment, the water delivery apparatus further comprises a target temperature register 410. In this variation of this alternative example embodiment, the user input device 310 generates a temperature-directive when a temperature-command is received from a user. Accordingly, the temperature directive is sent to the target temperature register 410, which modifies the value stored therein in response to the temperature directive received from the user input device 310.

According to one variation of this alternative example embodiment, the water delivery apparatus further comprises a target flow rate register 405. In this variation of this alternative example embodiment, the user input device 310 generates a flow-directive when a flow-command is received from a user. Accordingly, the flow-directive is sent to the target flow-rate register 405, which modifies the value stored therein in response to the flow-directive received from the user input device 310.

FIGS. 24A and 24B further illustrate that, according to one variation of this alternative example embodiment, the water delivery apparatus further comprises an emergency temperature comparator 415, which as heretofore described, generates a hot shut off signal 436 when the temperature of the mixed water exceeds a pre-established threshold. In this variation of this alternative example embodiment, the state machine 511 included in the temperature/flow rate controller 400, in response to receiving the hot shut off signal 436, causes the target flowrate register 405 to be cleared. In turn, this causes the hot control signal 426 to rapidly be reduced in order to shut off hot water flow into the mixing chamber 362.

FIGS. 18 and 24A also illustrate that, according to yet another variation of this alternative example embodiment, the water delivery apparatus further comprises an emergency temperature comparator 415, which as heretofore described, generates a shutoff mixed signal 446 when the temperature of the mixed water exceeds a pre-established threshold. In this variation of this alternative example embodiment, there is further included in the water delivery apparatus a mixed-water solenoid 385, which is installed between the mixing volume 362 and the mixed water output port (e.g. a faucet 399). In this variation of this alternative example embodiment, the mixed-water solenoid 385 response to the shutoff mixed signal 446 by substantially blocking water flow from the mixing volume 362 to the water output port.

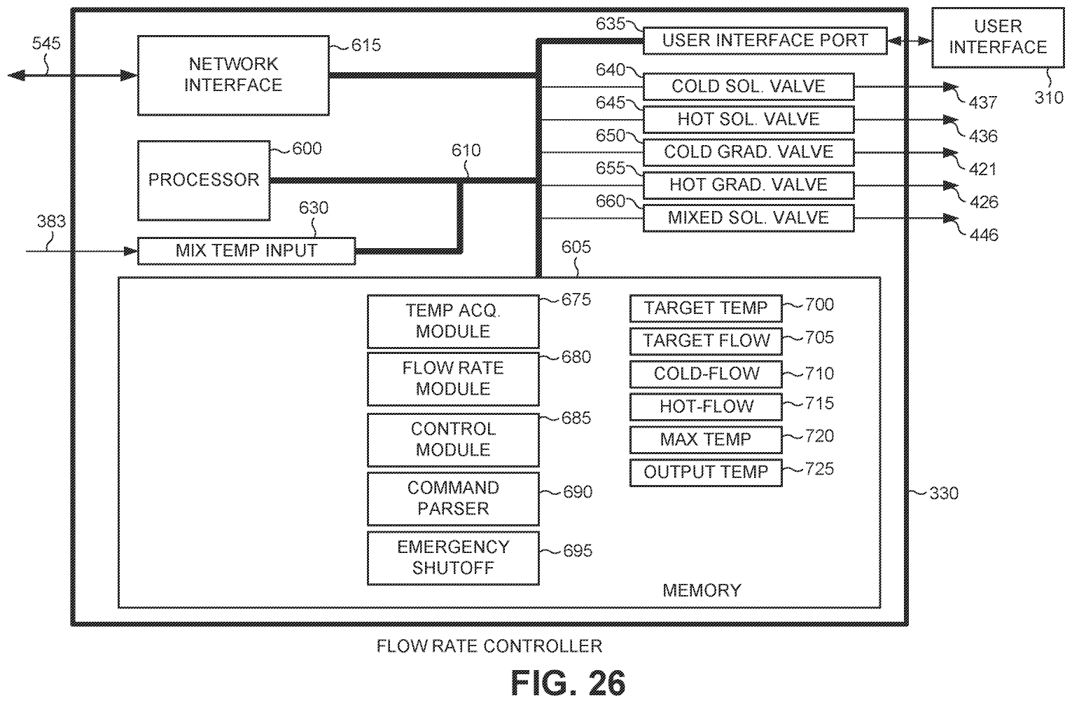

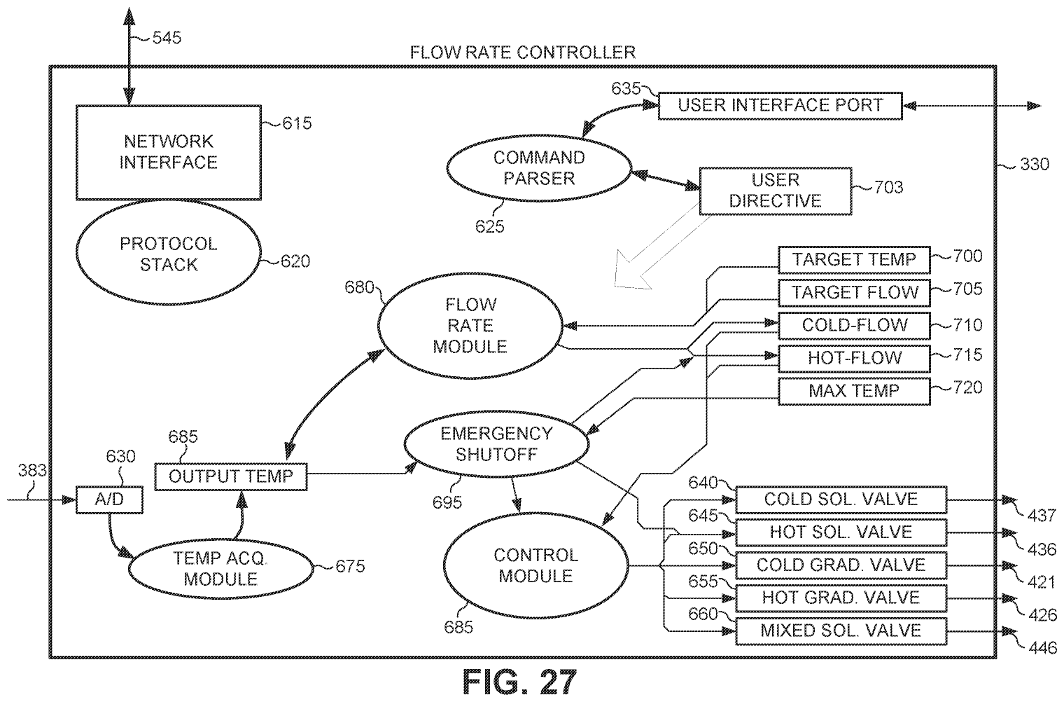

FIGS. 26 and 27 collectively constitute a block diagram of one alternative example embodiment of a flow rate controller that is based on a microprocessor. According to this alternative example embodiment of a water delivery apparatus, the flow rate controller 330 comprises a bus 610, a mixed temperature input port 630, a processor 600, and a memory 605. The bus 610 is used to communicate information from one element included in the flow rate controller 330 another element within the flow rate controller 330.

FIG. 27 shows that, according to this alternative example embodiment, the flow rate controller 330 also includes one or more instruction sequences stored in the memory 605 including a temperature acquisition module 675, a flow rate module 680 and a control module 685.

The reader is advised that the term “minimally causes the processor” and variants thereof is/are intended to serve as an open-ended enumeration of functions performed by the processor 600 as it executes a particular functional module (i.e. instruction sequence). As such, an embodiment where a particular functional module causes the processor 600 to perform functions in addition to those defined in the appended claims is to be included in the scope of the claims appended hereto.

The functional processes (and their corresponding instruction sequences) described herein enable delivery of temperature controlled water in accordance with the techniques, processes and other teachings of the present method. According to one alternative embodiment, these functional processes are imparted onto computer readable medium. Examples of such medium include, but are not limited to, random access memory, read-only memory (ROM), Compact Disk (CD ROM), Digital Versatile Disks (DVD), floppy disks, flash memory, and magnetic tape. This computer readable medium, which alone or in combination can constitute a stand-alone product, can be used to convert a general or special purpose computing platform into an apparatus capable of delivering temperature controlled water according to the techniques, processes, methods and teachings presented herein. Accordingly, the claims appended hereto are to include such computer readable medium imparted with such instruction sequences that enable execution of the present method and all of the teachings afore described.

According to this variation of this alternative example embodiment, the processor 600, as it executes the temperature acquisition module 675, is minimally caused to receive into the memory from the temperature input port 630 a current output temperature value. It should be appreciated that, according to this alternative example embodiment, the temperature input port 630 comprises an analog to digital (“A/D”) converter 630. Accordingly, the analog-to-digital converter 630 receives the mixed temperature signal 383 and generates a digital representation thereof. As such, the processor 600, as it continues to execute the temperature acquisition module 675, stores the output temperature value in an output temperature memory location 685. It should be appreciated that the output temperature memory location 685, as well as other memory locations, are stored in the memory 605 and are also referred to as variables.

The flow rate module 680 stored in the memory 605 of this variation of this alternative example embodiment, when executed by the processor, minimally causes the processor 600 to calculate and store in the memory a cold-side flow value and a hot-side flow value. It should be appreciated that, according to this variation of this alternative example embodiment, the processor 600 stores the cold-side flow value in a cold flow variable 710 and the processor 600 stores the hot-side flow value in a hot flow variable 715. The processor 600, as it executes the flowrate module 680 stored in the memory 605 of this variation of this alternative example embodiment calculates the hot-side flow value and the cold-side flow value in accordance with a target temperature value, which it obtains from the memory 605, and which is stored in a target temperature variable 700.

It should also appreciated that the processor 600 calculates the hot-side flow value and the cold-side flow value based on the mixed temperature value, which is stored in in the memory 605 in an output temperature variable 685. It should be appreciated that the processor 600 calculates the cold-side flow value and the hot-side flow value in accordance with the control law here in described, including a PID control structure. It should be appreciated that such PID control structures are commonly known and understood in the art and are not further described herein.

As the processor continues to execute this variation of the flowrate module 680, the processor 600 stores in the memory 605 a cold-side flow value and a hot-side flow value. It should be appreciated that, according to this variation of this alternative example embodiment, the processor 600 stores the cold-side flow value in a cold flow variable 710 and the processor 600 stores the hot-side flow value in a hot flow variable 715.

Once the processor 600 calculates the hot-side flow value and the cold-side flow value, the processor 600 then begins executing the control module 685. When executed by the processor 600, the control module 685 minimally causes the processor to retrieve a cold flow value and a hot flow value from the memory 605, wherein these values are stored in a cold flow variable 710 any hot flow variable 715. The processor 600 and then directs the cold flow value to the cold graduated valve control port 650 and directs the hot flow value to the hot graduated valve control port 655. The cold graduated valve control port 650 generates a cold graduated valve control signal 421 and the hot graduated valve port 655 generates a hot graduated valve control signal 426. These control signals are then directed to the hot-side graduated-valve 365 and the cold-side graduated-valve 360, respectively.

According to yet another variation of this alternative example embodiment, the flowrate controller 330 includes a user interface port 635 and a command parser module 625, which is stored in the memory 605. In this variation of this alternative example embodiment, the processor 600, as it executes the command parser module 625, is minimally caused to receive into the memory a temperature directive from a user interface 310. The processor 600 adjusts the target temperature value stored in the target temperature variable 700 according to the received temperature directive. In this manner, temperature directives, including at least one or more of an increase temperature directive and/or decrease temperature directive, I received by the processor 600 by way of the user interface port 635 and are used to affect changes in the target temperature value maintained in the memory 605, for example in the target temperature variable 700.

According yet another variation of this alternative example embodiment, the flowrate controller 330 includes a flow rate module 680 stored in the memory 605 which, when executed by the processor 600, minimally causes the processor to calculate and store in the memory a cold-side flow value and a hot-side flow value. It should be appreciated that, according to this variation of this alternative example embodiment, the processor 600 stores the cold-side flow value in a cold flow variable 710 and the processor 600 stores the hot-side flow value in a hot flow variable 715. In this variation of the flowrate controller 330, the flowrate module 680 also minimally causes the processor 600 to calculate the hot side flow value and the cold-side flow value on the target temperature value stored in the target temperature variable 700 and according to the target flowrate value, which is stored in the target flowrate variable 705, and according to the output temperature value stored in the output temperature variable 685.

FIG. 27 also illustrates that, according to yet another variation of this alternative example embodiment, the flowrate controller 330 further comprises a command parser 625, which is also stored in the memory 605. The command parser 625, when executed by the processor 600, minimally causes the processor to receive by way of the user interface 635 a user directive. It should be appreciated that, in this variation of this alternative example embodiment, the user directive is received from a user interface 310, as depicted in FIG. 26. When the processor 600 receives the user directive as it continues to execute the command parser 625, the processor 600 stores the user directive in a user directive variable 703, which is stored in the memory 605. The user directive variable 703 is available for access by the processor 600 as it executes other functional modules stored in the memory 605.

[Claim 50/51] it should be appreciated that, according to various illustrative embodiments herein described, the water delivery apparatus 300 includes a user input device 310 which is capable of receiving at least one or more of a start-flow-directive, a stop-flow-directive, an increase-flow-directive and/or a decrease-flow-directive. It should likewise be appreciated that, according to various illustrative example embodiments herein described, when the flowrate controller 330 receives a stop-flow-directive, the flowrate controller 330 generates a hot shut off signal 436 in conjunction with a cold shut off signal 437. The hot shut off signal 436 is directed to the hot-side solenoid-valve 355 and the cold shut off signal 437 is directed to the cold-side solenoid-valve 357. Accordingly, this causes disruption of water flow and discontinuance of water delivery at the mixed output, for example by way of a faucet 399. Conversely, when the flowrate controller 330 receives a start-flow-directive, the flowrate controller 330 deactivates, in a substantially contemporaneous manner, the hot shut off signal 436 and the cold shut off signal 437. It should be appreciated that this restores water flow and water delivery to the mixed output by way of the faucet 399.

In yet another variation of this alternative example embodiment, the user directive received by the processor 605 as it continues to execute the command parser 625 comprises a flow rate directive. When the processor 600 receives such a flow rate directive, the processor 600 adjusts the value of the target flowrate variable 705 as it continues to execute the command parser 625.

FIGS. 26 and 27 also illustrate that, according to yet another variation of this alternative example embodiment, the flowrate controller 330 further comprises an emergency shut off module 695. It should be appreciated that the emergency shut off module 695 is stored in the memory 605. The emergency shut off module 695 of this variation of this alternative example embodiment, when executed by the processor 600, minimally causes the processor 600 to compare a maximum temperature value, which is stored in a maximum temperature variable 720, which is itself stored in the memory 605, to the output temperature value stored in the output temperature variable 685. It should be appreciated that, according to this variation of this alternative example embodiment, the processor 600 executes the emergency shut off module 695 on a periodic basis. When the output temperature value stored in the output temperature variable exceeds the maximum temperature value, the processor 600, as it continues to execute the emergency shut off module 695, is further minimally caused to substantially reduce the hot-flow rate. The processor 600, as it continues to execute this variation of the emergency shut off module 695, accomplishes this by directly changing the value stored in the hot-flow variable 715, which is stored in the memory 605.

FIGS. 18, 26 and 27 also illustrate that, according to one alternative example embodiment, the water delivery apparatus further includes a hot-side solenoid-valve, which is installed between the hot-side input port 345 and the hot-side graduated valve 365. In this variation of this alternative example embodiment, the memory 605 further includes and has stored there in an emergency shut off module 695. In this variation of this alternative example embodiment, the processor 600 also executes the emergency shut off module 695 on a periodic basis. Akin to other embodiments herein described, this variation of the emergency shut off module 695, when executed by the processor 600, further minimally causes the processor to compare the maximum temperature value, which is stored in the maximum temperature variable 720 included in the memory 605, two a current output temperature, which is stored in an output temperature variable 685 included in the memory 605. When the comparison indicates that the current output temperature is greater than the maximum temperature, this variation of the emergency shut off module 695 further minimally causes the processor to activate the hot solenoid shut off valve 355, which the processor 600 accomplishes by sending a command to the hot solenoid valve output port 645. This generates a hot shut off signal 438, which is directed to the hot-side solenoid valve 355.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types.

While the present method and apparatus has been described in terms of several alternative and exemplary embodiments, it is contemplated that alternatives, modifications, permutations, and equivalents thereof will become apparent to those skilled in the art upon a reading of the specification and study of the drawings. It is therefore intended that the true spirit and scope of the claims appended hereto include all such alternatives, modifications, permutations, and equivalents.

What is claimed is:

1. A method for delivering temperature controlled water:
receiving a stream of cold water;
receiving a stream of hot water;
receiving a flow-directive from a user;
setting a target-temperature according to a default value;
setting a cold-side graduated-valve to a default flow rate;
setting a hot-side graduated-valve to a default flow rate;
allowing the stream of cold water to enter the cold-side graduated-valve;
allowing the stream of hot water to enter the hot-side graduated-valve;
directing into a mixing volume the water from the hot-side graduated-valve and the cold-side graduated-value;
sensing the temperature of the water in the mixing volume;
releasing mixed water from the mixing volume when the temperature is below a pre-established value; and
adjusting the flow-rate-setting of at least one or more of the cold-side graduated-valve and/or the hot-side graduated-valve according to the temperature of the water in the mixing volume and according to the target-temperature.

2. The method of claim 1 further comprising:
presenting to the user an indicator according to the temperature of the mixed water;
receiving a temperature-directive from the user; and
setting the target-temperature according to the temperature-directive.

3. The method of claim 1 further comprising:
preventing the stream of hot water from entering the hot-water graduated-valve when the temperature of the mixed water is greater than a pre-established value.

4. The method of claim 1 further comprising:
preventing water from exiting the mixing volume when at least one or more of the pressure and/or the actual-flow of the stream of cold water falls below a pre-established threshold.

5. The method of claim 1 further comprising:
preventing the stream of hot water from entering the hot-water graduated-valve and setting the flow rate of the cold-water graduated valve to a pre-established low-flow level and releasing the mixed water when the temperature of the mixed water is greater than a pre-established value.

6. The method of claim 1 further comprising:
wirelessly detecting a user identifier;
capturing an average flow rate during a first interval of time;
capture an average temperature of the mixed water during the first interval of time; and
communicating to a database the user identifier, the average flow rate, the average temperature of the mixed water and the length of the first interval of time.

7. The method of claim 6 further comprising communicating to the database the time elapsed from the first detection of the user identifier and the last detection of the user identifier.

8. The method of claim 6 further comprising:
capturing an image of the user; and
communicating to the database the captured image in association with the user identifier.

9. The method of claim 1 wherein receiving a flow-directive from a user comprises at least one or more of a start-flow-directive, a stop-flow-directive, an increase-flow-directive and/or a decrease-flow-directive.

10. The method of claim 1 wherein receiving a flow-directive from a user comprises perceiving a motion gesture expressed by a user.

11. The method of claim 1 wherein setting a target-temperature according to a default value comprises:
wirelessly detecting a user identifier; and
retrieving a default temperature value according to the user identifier.

12. The method of claim 1 further comprising:
capturing an average flow rate during a first interval of time;
capture an average temperature of the mixed water during the first interval of time;
present to the user an indication that the integral of flow rate and average temperature has met a minimum requirement.

13. The method of claim 1 wherein setting a cold-side graduated-valve to a default flow rate comprises:
sensing the temperature of at least one or more of the received stream of cold water and/or the received stream of hot water; and
setting the cold-side graduated-valve to a flow rate according to the temperature of at least one or more of the received stream of cold water and/or the received stream of hot water and according to the target temperature.

14. The method of claim 1 wherein setting a hot-side graduated-valve to a default flow rate comprises:
sensing the temperature of at least one or more of the received stream of cold water and/or the received stream of hot water; and
setting the hot-side graduated-valve to a flow rate according to the temperature of at least one or more of the received stream of cold water and/or the received stream of hot water and according to the target temperature.

15. The method of claim 1 further comprising:
receiving an amount of anti-microbial agent;
allowing the amount of anti-microbial agent to enter an anti-microbial agent valve; and
directing into the mixing volume the anti-microbial agent from the anti-microbial agent valve according to a dispense agent directive.

16. The method of claim 1 further comprising:
receiving a temperature-directive from the user; and
setting the target-temperature according to the temperature-directive.

17. The method of claim 16 wherein receiving a temperature-directive from the user comprises perceiving a motion gesture expressed by a user.

18. A water delivery apparatus that delivers temperature controlled water comprising:
cold-side solenoid valve that includes an input and an output;
input port for receiving cold water that is connected to the input of the cold-side solenoid valve;
cold-side graduated-valve that includes an output and an input that is connected to the output of the cold-side solenoid valve;
hot-side solenoid valve that includes an input and an output;
input port for receiving hot water that is connected to the input of the hot-side solenoid valve;
hot-side graduated-valve that includes an output and an input that is connected to the output of the hot-side solenoid valve;
mixing volume that includes a cold-side input that is connected to the output of the cold-side graduated-valve and a hot-side input that is connected to the output of the hot-side graduated-valve and an output;
output-temperature-sensor disposed so as to measure the temperature of water inside the mixing volume, and wherein the output-temperature-sensor generates an mixed-temperature-signal according to the temperature of the water in the mixing volume;
user-input-device that generates a flow-directive signal when a flow-directive is received from a user;
flow-rate-register that varies a target-flow-rate value according to a received flow-directive;
target-temperature register that provides an initial target-temperature value; and
flow-rate-controller that generates a cold-side-flow signal and a hot-side-flow signal according to the target-temperature value provided by the target-temperature register and according to the output-temperature-signal, and according to the target-flow-rate value, and wherein the cold-side-flow signal is directed to the cold-side graduated-valve, and wherein the hot-side-flow signal is directed to the hot-side graduated-valve.

19. The water delivery apparatus of claim 18 further comprising:
temperature-indicator that presents to a user an indication according to the output-temperature-signal, and wherein the user-input-device also generates a temperature-directive when a temperature-command is received from a user, and wherein the target-temperature register responds to the temperature-directive and modifies the value stored in the target-temperature register according to the temperature-directive.

20. The water delivery apparatus of claim 18 further comprising:
emergency-temperature-comparator that includes an input connected to the mixed-temperature-signal and an input connected to a pre-established maximum temperature value and which generates a hot-shutoff signal when the mixed-temperature-signal substantially exceeds the maximum temperature value, and wherein the hot-side solenoid responds to the hot-shutoff-signal by substantially blocking water flow from the hot water input port.
